(19) United States
(12) Patent Application Publication
(10) Pub. No.: US 2022/0155306 A1
(43) Pub. Date: May 19, 2022

(54) Biomarkers of Breast and Lung Cancer

(71) Applicant: University of Kentucky Research Foundation, Lexington, KY (US)

(72) Inventors: Andrew N. Lane, Lexington, KY (US); Teresa W. Fan, Lexington, KY (US); Richard M. Higashi, Lexington, KY (US)

(21) Appl. No.: 17/592,763

(22) Filed: Feb. 4, 2022

Related U.S. Application Data
(63) Continuation of application No. 15/908,500, filed on Feb. 28, 2018.
(60) Provisional application No. 62/464,891, filed on Feb. 28, 2017.
(60) Provisional application No. 62/608,180, filed on Dec. 20, 2017.

(51) Int. Cl. G01N 33/574; G16B 5/00; G16B 5/20

(52) U.S. Cl. CPC G01N 33/57488; G16B 5/00; G01N 33/57423; G01N 2405/02; G16B 5/20; G01N 2405/04; G01N 2405/06; G01N 33/57415

(57) ABSTRACT

Provided herein are methods of detecting lipids in humans suspected of having cancer, in particular detecting lipids in samples from a human suspected of having breast or lung cancer.

### RELATED APPLICATIONS

This application is a continuation from U.S. patent application Ser. No. 15/908,500, filed Feb. 28, 2018, which claims priority from U.S. Provisional Application Ser. No. 62/464,891, filed Feb. 28, 2017, and 62/608,180, filed Dec. 20, 2017, the entire disclosures of which are incorporated herein by this reference.

### GOVERNMENT INTEREST

This invention was made with government support under grants P1 CA163223, P3 CA177558, 1U24DK097215, and R3 CA222449 awarded by the National Institutes of Health (NIH). The government has certain rights in the invention.

### TECHNICAL FIELD

The presently-disclosed subject matter relates to biomarkers of breast and lung cancer and methods of detecting the biomarkers for determining the presence or absence of cancer.

### INTRODUCTION

Blood plasma contains small vesicles of different sizes, operationally defined as lipidic microvesicles and exosomes, which are shed from cells of most tissues. Exosomes are secreted by living cells and carried throughout the body via blood and lymph, and are found in body fluids such as urine, and saliva. Exosomes serve as transporters of bioactive molecules (nucleic acids, proteins, lipids) and can be reabsorbed by cells at distant sites/tissues. In doing so, exosomes transport cellular components from one site in the body to another. As such exosomes are a means of delivering specific bioactive molecules from one tissue site to another. Microvesicles are larger and more heterogeneous lipidic particles that are shed from red blood corpuscles and potentially dying cells. They may represent a form of “debris” rather than functional entities, as are exosomes. Nevertheless, they might have value for diagnostic or prognostic purposes.

The steady state level of circulating exosomes in adult human plasma has been estimated at around 1 mg/ml in healthy individuals, and as much as three times higher in individuals who have certain carcinomas, including those of the breast. Over the past decade, significant efforts have been made to elucidate the function of exosomes as related to basic immunology as well as to cancer biology. In particular, research has focused on determining differences in exosome content among healthy and diseased cells. In line with this, a major goal has been to utilize exosome components as biomarkers for various diseases. Recently, it has been shown that there is indeed a difference in lipid composition of exosomes between healthy individuals and those with certain cancers. This, in turn, has prompted efforts to screen and identify lipid panels among prostate cancer patients compared with serum from healthy controls.

A long-standing goal in cancer biology has been to develop tests that can detect cancer early, accurately predict prognosis, and facilitate selection among therapies. For example, current breast cancer screening relies primarily on physical exams as well as mammography. Even when performed regularly, these methods do not ensure detection of cancer at an early stage when treatment is most effective. The use of biomarker tests could also greatly facilitate and accelerate the development of targeted cancer therapies by helping companies choose the most promising drug candidates and by identifying patients that are most likely to benefit from a given therapy.

Lung cancer is by far the leading cause of cancer deaths in the U.S. with an estimated 224,390 new cases and 158,080 deaths in 2016. Kentucky now leads the nation both in terms of lung cancer incidence and mortality, with the Appalachian population posting even higher incidence and mortality rates. Patients with early stage lung cancer have the best prognosis with surgical removal of the tumor, but the disease is often asymptomatic, and there are no effective screening methods for early detection of lung cancer in at-risk populations. Consequently, most lung cancer patients are diagnosed at advanced stages due to the silent nature of the early stage disease. Although the five-year survival rate of localized lung cancer is ˜55% with proper surgical intervention, that of advanced stage disease drops to ˜4%. Presently, there is no robust low-cost screening method for detecting asymptomatic early stage lung cancer. Current imaging or cytology-based methods are impractical for screening at-risk populations for lung cancer, as they are not sufficiently accurate, cost-effective or non-invasive. Although low dose helical CT screens have recently been reported to decrease lung cancer mortality by 20% in comparison to chest x-ray screening, there remains a high false positive rate. Thus, techniques to detect and reliably screen lung cancer at its earliest stage in at-risk populations are urgently needed to improve survival and quality of life for lung cancer patients.

Non-small cell lung cancer (NSCLC) is the dominant form (ca. 85%) of lung cancer, and comprises many subtypes with different sets of oncogenic drivers such as mutant KRAS, EGFR, LKB1, EML4-ALK (adenocarcinomas), PIK3CA, NRF2 (squamous cell carcinomas), cMYC overexpression and inactivation of TP53 via mutations (both subtypes), and numerous other genetic aberrations yet to be functionally defined. It is becoming clear that one of the key functions of these oncogenic drivers lies in reprogramming specific metabolic events in cancer cells to promote their proliferation, survival and metastasis. Thus, metabolic reprogramming in cancer has been recently recognized as a hallmark of cancer. However, the global metabolic networks, and lipidomics in particular, modulated by these drivers and/or other undefined genetic aberrations are poorly characterized in NSCLC. It would be advantageous to provide a screening and/or early indicator for cancers, particularly lung cancer and breast cancer.

### SUMMARY

The presently-disclosed subject matter meets some or all of the above-identified needs, as will become evident to those of ordinary skill in the art after a study of information provided in this document. To address the needs in the art, the presently disclosed subject matter includes biomarkers of breast and lung cancer.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

Disclosed herein are methods for detecting or determining lipid amounts in a human suspected of having breast, lung cancer. In some embodiments, the methods disclosed include the steps of providing a sample comprising a bodily fluid or treatment thereof from the human suspected of having breast cancer, breast disease, or lung cancer; and determining the lipid amounts in a lipid set in the sample. In some embodiments, the lipid set comprises at least five lipids, at least ten lipids, or at least fifteen lipids.

In some embodiments, the lipid set comprises a lipid from the class of TG-triacylglycerol, DG—diacylglycerol, PIP2-phosphatidyl inositol bisphosphate, PIP-phosphatidyl inositol phosphate, MGDG—Monogalactosyldiacylglycerol, MGMG—monogalactosylmonoacylglycerol, MG—monoacylglycerol, PC—phosphatidyl choline, PS—phosphatidyl serine, PE—phosphatidyl ethanolamine, PG—phosphatidyl glycerol, dMePE—dimethylphosphatidyl ethanolamine, So—Sphingosine, LPG—lyso phosphatidyl glycerol, LdMePE—lyso dimethylphosphatidyl ethanolamine, LPC—lyso phosphatidyl choline, LPE—lyso phosphatidyl ethanolamine, LPI—lysi phosphatidyl inositol, Pet—phosphoethanolamine, Cer—ceramide, CerG2GNAc1—neutral glycosphingolipid, LPA—lyso phosphatidic acid, PA—phosphatidic acid, PI—phosphatidyl inositol, cPA—cyclic phosphatidyl acid, LPEt—lysophosphoethanoloamine, phSM—phosphosphingomyelin, PMe—phosphomethanol, cholesterol esters (CE), triacylglyceride (TAG), lysophosphatidylcholine (Lyso-PC), lysophosphatidylcholine-plasmalogen (LysoPC—pmg), phosphatidyl choline (PC), and sphingomyelin (SM).

In some embodiments, the one or more lipids are selected from PIP2, PIP, MGDG, MGMG, Pet, CerG2GNAc1, cPA, LPet, phSM, and PMe.

In some embodiments, the one or more lipids are selected from PIP2 (42:7), PIP2 (48:7), PIP2 (46:7), PIP2 (41:0), PIP (55:6), PIP (29:3), PIP (29:2), PIP (30:6), PIP (48:8), PIP (46:5), MGDG (23:6), MGDG (45:10), MGDG (46:10), MGDG (42:6), MGDG (27:7), MGDG (37:8), MGDG (26:1), MGDG (27:1), MGDG (7:0), MGDG (33:15), MGDG (13:6), MGMG (23:10), MGMG (11:3), Pet (28:2), Pet (31:2), Pet (22:2), CerG2GNAc1 (34:2), cPA (18:2), cPa (16:0), LPet (30:4), phSM (27:4), phSM (27:1), phSM (28:1), phSM (28:0), phSM (28:4), PMe (31:23), and PMe (32:2).

In some instances, the lipid set further includes one of more lipids selected from the group consisting of TG (68:5), TG (68:6), TG (22:6), TG (51:0), TG (67:6), TG (71:6), TG (77:6), TG (46:4), TG (58:6), TG (56:6), TG (75:6), TG (52:2), TG (50:0), TG (42:1), TG (43:2), TG (34:2), TG (35:2), DG (24:2), DG (38:6), DG (53:6), DG (17:0), DG (21:0), DG (28:0), DG (40:8), DG (38:8), MG (14:0), MG (18:0), PC (34:7), PC (33:0), PC (32:0), PC (34:6), PC (28:0), PC (28:3), PC (25:0), PC (28:2), PS (23:0), PS (37:2), PE (29:0), PE (31:2), PE (31:3), PE (30:8), PE (30:3), PE (28:0), PG (32:0), PG (37:4), dMePE (28:1), dMePE (8:0), dMePE (29:3), dMePE (29:2), dMePE (28:2), dMePE (28:3), dMePE (26:0), So (d16:1), LPG (12:0), LPG (15:0), LdMePE (27:0), LdMePE (28:3), LdMePE (27:4), LdMePE (29:3), LdMePE (26:0), LdMePE (28:4), LPC (26:0), LPC (25:0), LPC (27:3), LPC (28:3), LPE (29:0), LPE (28:0), LPE (30:3), LPE (8:0), LPI (16:1), Cer (24:1), Cer (26:0), Cer (24:0), LPA (33:4), LPA (32:4), PA (23:4), PA (33:3), PA (32:3), PA (32:4), PA (33:2), PA (24:2), PA (32:2), PI (51:8).

The methods disclosed herein, in some instances include detection in a human suspected of having lung cancer. In some embodiments, the lung cancer is selected from small cell (SCLC) and non-small cell type (NSCLC) lung cancer. In some embodiments, the methods of determining amounts of lipids are performed in a sample from a human suspected of having breast cancer or breast disease. In some embodiments, the breast cancer is selected from DCIS, LCIS, invasive ductal and lobular, inflammatory (triple negative) and metastatic disease. In some embodiments, the breast disease is inflammatory breast disease.

A method of detecting lung cancer in a subject is disclosed herein, which, in some embodiments, includes the steps of isolating exosomes from a sample of whole blood, or a blood component, and detecting lipids in the isolated exosomes in a lipid set. In some embodiments the lipids can include one or more of PC (18:2/18:1), PC (18:2/18:0), PC (22:6/16:0), PC (18:2/16:0), SM (18:1/16:0), PC (20:3/18:0), PC (20:4/16:0), PC (22:5/16:0), CE (20:4), TAG (18:1/18:2/16:2), SM (18:1/24:1), PC (18:1/18:0), PC (16:0/16:0), TAG (18:2/16:0/20:4), LysoPC (16:0), or LysoPC-pmg (12:0). In some embodiments, the methods disclosed herein are capable of distinguishing whether a subject does not have cancer, has early stage cancer, or has late stage cancer. In some embodiments, the methods allow for the distinguishing subjects as having early stage or late stage cancer. In some embodiments, the cancer is lung cancer, in some embodiments, the cancer is non-small cell lung cancer.

In some embodiments, the sample is a bodily fluid. In some instances the bodily fluid is blood serum or plasma. In some embodiments, the sample comprises a lipid exosomal fraction, microvesicle fraction, or a combination thereof.

One method of evaluating a blood sample from a patient, include the steps of obtaining the blood sample from the patient; isolating an exosomal fraction from the blood sample; measuring levels for two or more lipids in the exosomal fraction to generate test data; applying an algorithm to the lipid levels of step measured in the exosomal fraction that correlates the lipid levels measured with lipid data obtained from a plurality of samples, where the plurality of samples include samples from patients with cancer such as non-small cell lung cancer (NSCLC) and without cancer. In some embodiments, the algorithm is a trained algorithm trained by the lipid data obtained from the plurality of samples.

Based on the correlation of lipid levels, the method can further include identifying the patient as having an increased probability of early stage cancer, identifying the patient as having an increased likelihood of late stage cancer, or identifying the patient as normal, identifying the patient as having increased probability of cancer, identifying the patient as having an increased probability of having a benign condition. In some embodiments, the correlating uses lipid data of at least three of the following lipids: PC (18:2/18:1), PC (18:2/18:0), PC (22:6/16:0), PC (18:2/16:0), SM (18:1/16:0), PC (20:3/18:0), PC (20:4/16:0), PC (22:5/16:0), CE (20:4), TAG (18:1/18:2/16:2), SM (18:1/24:1), PC (18:1/18:0), PC (16:0/16:0), TAG (18:2/16:0/20:4), LysoPC (16:0), and LysoPC-pmg (12:0). In some embodiments, the method can further include treating said patient on the basis of the identification.

### BRIEF DESCRIPTION OF THE DRAWINGS

Illustrative aspects of embodiments of the present invention will be described in detail with reference to the following figures wherein:

FIG. 1 includes a plot showing a “Power Plot” of positive mode lipids from exosomes. The Area under the ROC Curve (AUC), which is a measure of overall accuracy, was calculated for each model, and plotted against the sample size (taken at random from the total data set), and extrapolates to an AUC of ca. 0.96, with n=104; recent results do not support this high AUC.

FIG. 2A shows plots of lipid discriminator importance utilizing Random Forests and FIG. 2B plots lipid discrimintator importance utilizing Orthogonal Partial Least Squares Discriminant Analysis (OPLS-DA).

FIG. 3 includes AUC versus sample size for Random Forest analysis of breast samples.

FIG. 4 includes an OPLS-DA Scatter plot of all breast cancer, disease and healthy plasma samples of exosomal lipids in FT-MS [+] ion mode.

FIG. 5 includes left hand side of the SimcaP VIP plot of exosomal healthy, breast cancer and benign breast disease samples.

FIG. 6A includes Gini importance of lipid features discerned from the plasma exosomal MS data of normal and lung cancer subjects including the Gini importance of all 430 assigned lipid features in the entire exosomal MS data set; and FIG. 6B The Gini importance of the top 16 lipid features averaged over 500 tree predictors. Error bars represent standard deviations.

FIG. 7 includes a Random Forest (RF) proximity plot of the lung cancer exosome mass data set. The RF analysis was performed as described in the Example 3 Methods with mass ion intensities of each sample normalized to the summed intensities of lipid features that were non-zero in 20% of all samples. The top 16 lipid features from FIG. 8 were used for the classification with 5-fold cross validation replicated 500 times.

FIG. 8 includes boxplots of intensities distribution of top RF-selected 16 lipid features for normal, early, and late-stage lung cancer subjects. The top 16 lipid features were selected according to their Gini importance values (cf. FIG. 6b) These features were assigned to molecular formulae (as shown) based on their accurate masses using PREMISE and validated as specific lipid species via their MS² fragmentation patterns (cf. Table 7).

FIG. 9 includes the LASSO predicted score of the lung cancer exosome mass data set. For each sample, predicted probability scores of belonging to each of the normal, early stage, and late stage groups were calculated based on a 7-feature model obtained from LASSO. A higher score indicates a higher probability of belonging to the group.

FIG. 10 shows the boxplots of intensities distribution of LASSO-selected 7 lipid features for normal, early, and late-stage lung cancer subjects.

These features were assigned to molecular formulae (as shown) based on their accurate masses using PREMISE and three of which were validated as specific lipid species via their MS² fragmentation patterns (cf. Table 7).

FIG. 11A provides exploratory PCA and FIG. 11B provides OPLSDA of plasma exosomal MS data acquired from normal, early and late-stage NSCLC subjects, performed using the top 16 lipid features on the same plasma exosomal MS data as in FIG. 6 via the SIMCAP software package. These analyses provided no clear separation of normal from lung cancer subjects and revealed only a few outliers in the dataset; FIG. 11C provides PCA with all 1102 features from 130 blood samples (39 healthy samples, 44 early and 47 late NSCLC samples) and 26 solvent samples acquired at the same time of the blood samples. Features that have non-zero intensity values in any of the solvent samples were removed as solvent impurities before feature selection and classification. The larger x-axis and y-axis ranges arose because the top 2 principal components transferred from the 1102 features data have larger variance than that transferred from 16 features data.

FIG. 12A includes Receiver Operating Characteristic curves (ROCs) for Random Forest-based classification of normal, early, and late-stage lung cancer subjects obtained from the RF analysis in FIG. 7 with area under the curve (AUC) calculated as shown for normal versus early-stage, FIG. 12B shows the ROC for normal versus late-stage, and FIG. 12C shows the ROC for early versus late-stage NSCLC subjects.

FIG. 13A includes size distribution analysis of isolated plasma exosomes of sample from a subject diagnosed with NSCLC and FIG. 13B includes the analysis for a sample from a healthy individual. Exosomes were isolated from patient blood plasma as described in the Methods of Example 2 and diluted with PBS to give a suitable number density for size analysis and counting using the Nanosight 300. % scans were recorded and averaged. Error bars indicated+/−1 s.d. of the mean. The numbers in the graph are the mode values of each peak.

FIG. 14A includes representative UHR-FTMS spectra of exosomal lipids of MS1 spectrum from a lung cancer patient shows the distribution of monoisotopic lipid features (accurate mass as values in outlined boxes) from Table 7. The region from 690-910 m/z is plotted with 50× expansion on the intensity scale, and the labels in black are m/z values, below which are resolutions at the corresponding m/z, as displayed by Xcalibur. Lipid features (as defined in the text) were used to annotate this figure since they were actually used for classification in this study, instead of the molecular formulae and lipid names in Table 7; FIG. 14B shows a Selected-range MS2 spectrum of the 424.2823 m/z precursor from the same sample, showing loss of a C12H23 group and generation of phosphocholine fragment. MS2 parameters were as described in Methods except that MS1 used a resolving power of 500,000 (at 200 m/z) and isolation window of 0.4 m/z to better define the precursors; the collision energy used intentionally retained some molecular ion in the MS2 mode, as evident here.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom.

The presently-disclosed subject matter meets some or all of the above-identified needs, as will become evident to those of ordinary skill in the art after a study of information provided in this document. To avoid excessive repetition, this Description does not list or suggest all possible combinations of such features.

Mention of one or more representative features of a given embodiment is likewise exemplary. Such an embodiment can typically exist with or without the feature(s) mentioned; likewise, those features can be applied to other embodiments of the presently-disclosed subject matter, whether listed or not.

Disclosed herein is a screening assay to analyze lipid content of exosomes isolated from cancer samples verses healthy controls, distinctions among samples sets, and methods for screening and identifying distinct lipid panels from exosomes for different cancers. A significant number of lipids have been identified which can be utilized as early predictors/biomarkers for cancer. These exosome-derived lipids can be utilized as new, highly sensitive, minimally invasive biomarker screens for various types of cancer including breast and lung cancers. Such distinct lipid profiles from exosomes isolated from cancer plasma samples can distinguish cancer from healthy controls. Analyses disclosed herein have identified discernable lipid patterns among healthy controls, breast cancer (BrCa) samples, as well as benign cancer samples. Lipid profiles from exosomes of healthy controls and those obtained from lung samples have also been conducted and the distinctive lipid biomarkers can be used as diagnostic screens in clinical laboratories. The methods disclosed herein, for example, allow for early stage detection of breast and lung cancers.

Some embodiments of the invention include methods for detecting the presence or absence of one or more cancer types by determining the amount of lipids in a lipid set in a sample. The sample can be a bodily fluid (or treatment thereof) from an animal. In some instances, the sample (e.g., a bodily fluid extract) comprises a concentration of lipid exosomes that is higher or lower than normally found in a bodily fluid from a healthy or non-cancerous subject. The lipid amounts in the lipid set are analyzed using a predictive model to determine the presence or absence of one or more cancer types based on the exosomal lipid pattern (e.g. the relative increases and/or decreases in the lipid set when comparing the lipid exosomal amounts of a control and subject suspected of having a condition).

In some embodiments, the present invention provides a method of diagnosing cancer that gives a specificity or sensitivity that is greater than 70% using the subject methods described herein, wherein the gene expression product levels are compared between the biological sample and a control sample; and identifying the biological sample as cancerous if there is a difference in the gene expression levels between the biological sample and the control sample at a specified confidence level. In some embodiments, the specificity and/or sensitivity of the present method is at least 70%, 75%, 80%, 85%, 86%, 87%, 88%, 89%, 90%, 91%, 92%, 93%, 94%, 95%, 96%, 97%, 98%, 99%, or more.

A method for determining the presence or absence of at least one cancer type in an animal is disclosed herein that includes the steps of determining amounts of lipids in a lipid set in a sample from the animal, and determining the presence or absence of at least one cancer type in the animal with a predictive model, wherein the lipid amounts of lipids in the lipid set comprise an input of the predictive model. The sample includes a bodily fluid or treatment thereof; and the at least one cancer type is selected from the group consisting of carcinomas, breast cancer, lung cancer, cancers that can alter the regulation or activity of Pyruvate Carboxylase, and tumors associated with any of the aforementioned cancer types. In some embodiments, the cancer is selected from breast or lung cancer. In some embodiments, the methods of detecting lipid sets can distinguish breast cancer from benign breast disease. In some embodiments, the methods aid in distinguishing the type of cancer or advancement of cancer. For example, in some embodiments, the methods can detect DCIS, and invasive carcinoma.

In some embodiments, methods of the presently-disclosed subject matter make use of biomarkers for detecting the presence or absence of a cancer. In some embodiments, the cancer can be breast, small cell lung, or squamous lung. In some embodiments, the cancer can be: breast or lung (including small cell (SCLC) and non-small cell type (NSCLC)). In some embodiments, the breast cancer is identified at different stages, for example, DCIS, LCIS, invasive ductal and lobular, inflammatory (triple negative) and metastatic disease. In other embodiments, the method can distinguish between breast cancer and benign breast lesions. In some embodiments the method detects or differentiates between benign lung disease (e.g. COPD, emphysema) versus early stage NSCLC versus late stage NSCLC. A distinct advantage of this method is to provide a non-invasive testing that can facilitate early detection in the presence or absence of other criteria, including imaging.

In some embodiments, the lipid set comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more lipids. In some embodiments, the lipid set comprises at least 1, 2, 3, 4, 5, 6, 7, 8, 9, 10, 11, 12, 13, 14, 15, 16, 17, 18, 19, or 20 lipids.

In some embodiments, lipids, which can be extracted from the exosomal fraction of sample, may provide increased accuracy of a genetic disorder or cancer diagnosis through the use of multiple lipids in low quantity and quality, and statistical analysis using algorithms of the present invention. In particular, the present invention provides, but is not limited to, methods of diagnosing, characterizing and classifying exosomal lipid profiles associated with lung and breast cancers. The present invention also provides algorithms and methods for characterizing and classifying early and late stage cancers, as well as benign conditions, and kits and compositions useful for the application of said methods.

In some embodiments, a predictive model is used and comprises one or more of dimension reduction method, clustering method, machine learning method, principal components analysis, soft independent modeling of class analogy, partial least squares regression, orthogonal least squares regression, partial least squares discriminant analysis, orthogonal partial least squares discriminant analysis, mean centering, median centering, Pareto scaling, unit variance scaling, orthogonal signal correction, integration, differentiation, cross-validation, or receiver operating characteristic curves, least absolute shrinkage and selection operator analysis (LASSO) and random forest.

Raw lipid levels and data may in some instances be improved through the application of algorithms designed to normalize and or improve the reliability of the data. In some embodiments of the present invention the data analysis requires a computer or other device, machine or apparatus for application of the various algorithms described herein due to the large number of individual data points that are processed. A machine learning algorithm refers to a computational-based prediction methodology, also known to persons skilled in the art as a classifier, employed for characterizing a lipid profile. The signals corresponding to certain lipid levels in a set are typically subjected to the algorithm in order to classify the lipid profile. Supervised learning generally involves training a classifier to recognize the distinctions among classes and then testing the accuracy of the classifier on an independent test set. For new, unknown samples the classifier can be used to predict the class in which the samples belong. The classification can, in some embodiments, be performed using random forest or LASSO algorithms.

The results of the profiling may be classified into one of the following: benign (free of a cancer, disease, or condition), malignant (positive diagnosis for a cancer, disease, or condition), or non-diagnostic (providing inadequate information concerning the presence or absence of a cancer, disease, or condition). In some cases, a diagnostic result may further classify the type of cancer, disease or condition. In other cases, a diagnostic result may indicate a certain grade or stage of a particular cancer disease or condition. In some embodiments of the present invention, results are classified using a trained algorithm.

In some embodiments, the bodily fluid is selected from the group consisting of plasma vomit, cerumen, gastric juice, breast milk, mucus, saliva, sebum, semen, sweat, tears, vaginal secretion, blood serum, aqueous humor, vitreous humor, endolymph, perilymph, peritoneal fluid, pleural fluid, cerebrospinal fluid, blood, plasma, nipple aspirate fluid, urine, stool, and bronchioalveolar lavage fluid. In some embodiments, the bodily fluid is plasma. In some embodiments, the lipids are exosomal or microvesicle lipids.

In some embodiments, the lipid set comprises a lipid from the class of TG—triacylglycerol, DG—diacylglycerol, PIP2—phosphatidyl inositol bisphosphate, PIP—phosphatidyl inositol phosphate, MGDG—Monogalactosyldiacylglycerol, MGMG—monogalactosylmonoacylglycerol, MG—monoacylglycerol, PC—phosphatidyl choline, PS—phosphatidyl serine, PE—phosphatidyl ethanolamine, PG—phosphatidyl glycerol, dMePE—dimethylphosphatidyl ethanolamine, So—Sphingosine, LPG—lyso phosphatidyl glycerol, LdMePE—lyso dimethylphosphatidyl ethanolamine, LPC—lyso phosphatidyl choline, LPE—lyso phosphatidyl ethanolamine, LPI—lysi phosphatidyl inositol, Pet—phosphoethanolamine, Cer—ceramide, CerG2GNAc1—neutral glycosphingolipid, LPA—lyso phosphatidic acid, PA—phosphatidic acid, PI—phosphatidyl inositol, cPA—cyclic phosphatidyl acid, LPEt—lysophosphoethanoloamine, phSM—phosphosphingomyelin, and PMe—phosphomethanol.

In some embodiments, the lipid set comprises at least 3, at least 5, at least 7, at least 10, at least 15, at least 20, or at least 50 lipids. In some embodiments, the one or more lipid in the lipid set are further selected from the group consisting of: TG (68:5), TG (68:6), TG (22:6), TG (51:0), TG (67:6), TG (71:6), TG (77:6), TG (46:4), TG (58:6), TG (56:6), TG (75:6), TG (52:2), TG (50:0), TG (42:1), TG (43:2), TG (34:2), TG (35:2), DG (24:2), DG (38:6), DG (53:6), DG (17:0), DG (21:0), DG (28:0), DG (40:8), DG (38:8), PIP2 (42:7), PIP2 (48:7), PIP2 (46:7), PIP2 (41:0), PIP (55:6), PIP (29:3), PIP (29:2), PIP (30:6), PIP (48:8), PIP (46:5), MGDG (23:6), MGDG (45:10), MGDG (46:10), MGDG (42:6), MGDG (27:7), MGDG (37:8), MGDG (26:1), MGDG (27:1), MGDG (7:0), MGDG (33:15), MGDG (13:6), MGMG (23:10), MGMG (11:3), MG (14:0), MG (18:0), PC (34:7), PC (33:0), PC (32:0), PC (34:6), PC (28:0), PC (28:3), PC (25:0), PC (28:2), PS (23:0), PS (37:2), PE (29:0), PE (31:2), PE (31:3), PE (30:8), PE (30:3), PE (28:0), PG (32:0), PG (37:4), dMePE (28:1), dMePE (8:0), dMePE (29:3), dMePE (29:2), dMePE (28:2), dMePE (28:3), dMePE (26:0), So (d16:1), LPG (12:0), LPG (15:0), LdMePE (27:0), LdMePE (28:3), LdMePE (27:4), LdMePE (29:3), LdMePE (26:0), LdMePE (28:4), LPC (26:0), LPC (25:0), LPC (27:3), LPC (28:3), LPE (29:0), LPE (28:0), LPE (30:3), LPE (8:0), LPI (16:1), Pet (28:2), Pet (31:2), Pet (22:2), CerG2GNAc1 (34:2), Cer (24:1), Cer (26:0), Cer (24:0), LPA (33:4), LPA (32:4), PA (23:4), PA (33:3), PA (32:3), PA (32:4), PA (33:2), PA (24:2), PA (32:2), PI (51:8), cPA (18:2), Cpa (16:0), LPet (30:4), phSM (27:4), phSM (27:1), phSM (28:1), phSM (28:0), phSM (28:4), PMe (31:23), and PMe (32:2).

In some embodiments, the lipids are selected from PC (18:2/18:1), PC (18:2/18:0), PC (22:6/16:0), PC (18:2/16:0), SM (18:1/16:0), PC (20:3/18:0), PC (20:4/16:0), PC (22:5/16:0), CE (20:4), TAG (18:1/18:2/16:2), SM (18:1/24:1), PC (18:1/18:0), PC (16:0/16:0), TAG (18:2/16:0/20:4), LysoPC (16:0), and LysoPC-pmg (12:0).

The presently-disclosed subject matter includes lipid sets that are useful as biomarkers in the diagnosis of cancer. A lipid set is defined to include one or more lipids. The term lipid, as used herein, is defined as a collection of one or more isomers. For example, PC (36:1) is a lipid and is the collection of one or more of the phosphatidylcholine isomers that have 36 carbons in the acyl chain and one double bond in either of the two acyl chains; these isomers have identical molecular weights. Although the term lipid can encompass the entire collection of isomers, the sample may, in fact, have only one isomer, several isomers, or any number of isomers less than the total number of all possible isomers in a collection. Accordingly, lipid can refer to one or more of the isomers that make up the entire collection of possible isomers. Reference to lipid amount (and similar phrases, such as amounts of lipids or amount of a lipid) is defined to encompass an absolute amount of a lipid (e.g. in mmoles) or a relative amount of a lipid (e.g., in % relative intensity). Lipids can be designated according notation XXX (YY:ZZ) where XXX is the abbreviation for the lipid group (in many instances indicating the lipid headgroup) as provided, for example in conjunction with Table 5, YY is the number of carbons in the acyl chain, and ZZ is the number of double bonds in the acyl chains.

The presently disclosed subject matter is also directed to a biomarker for lung or breast cancer comprising: a lipid set derived from the exosomes of a human bodily fluid; the lipid set comprising 15 or more lipids from the group consisting of: TG (68:5), TG (68:6), TG (22:6), TG (51:0), TG (67:6), TG (71:6), TG (77:6), TG (46:4), TG (58:6), TG (56:6), TG (75:6), TG (52:2), TG (50:0), TG (42:1), TG (43:2), TG (34:2), TG (35:2), DG (24:2), DG (38:6), DG (53:6), DG (17:0), DG (21:0), DG (28:0), DG (40:8), DG (38:8), PIP2 (42:7), PIP2 (48:7), PIP2 (46:7), PIP2 (41:0), PIP (55:6), PIP (29:3), PIP (29:2), PIP (30:6), PIP (48:8), PIP (46:5), MGDG (23:6), MGDG (45:10), MGDG (46:10), MGDG (42:6), MGDG (27:7), MGDG (37:8), MGDG (26:1), MGDG (27:1), MGDG (7:0), MGDG (33:15), MGDG (13:6), MGMG (23:10), MGMG (11:3), MG (14:0), MG (18:0), PC (34:7), PC (33:0), PC (32:0), PC (34:6), PC (28:0), PC (28:3), PC (25:0), PC (28:2), PS (23:0), PS (37:2), PE (29:0), PE (31:2), PE (31:3), PE (30:8), PE (30:3), PE (28:0), PG (32:0), PG (37:4), dMePE (28:1), dMePE (8:0), dMePE (29:3), dMePE (29:2), dMePE (28:2), dMePE (28:3), dMePE (26:0), So (d16:1), LPG (12:0), LPG (15:0), LdMePE (27:0), LdMePE (28:3), LdMePE (27:4), LdMePE (29:3), LdMePE (26:0), LdMePE (28:4), LPC (26:0), LPC (25:0), LPC (27:3), LPC (28:3), LPE (29:0), LPE (28:0), LPE (30:3), LPE (8:0), LPI (16:1), Pet (28:2), Pet (31:2), Pet (22:2), CerG2GNAc1 (34:2), Cer (24:1), Cer (26:0), Cer (24:0), LPA (33:4), LPA (32:4), PA (23:4), PA (33:3), PA (32:3), PA (32:4), PA (33:2), PA (24:2), PA (32:2), PI (51:8), cPA (18:2), Cpa (16:0), LPet (30:4), phSM (27:4), phSM (27:1), phSM (28:1), phSM (28:0), phSM (28:4), PMe (31:23), and PMe (32:2).

In some embodiments, the lipid sets are biomarkers for breast cancer. In some embodiments, there are at least 10, 15 or 18 lipid sets. In some embodiments, the lipid sets include: LPMe (18:0e), LPMe (16:0e), FA (18:0), MGMG (9:5), FA (15:0), LPG (29:4), PG (8:0e/21:4) PG (8:0p/21:3)-H 26 1.0000000, MGMG (29:8), PG (8:0p/24:7), LPA (32:4), LPEt (30:4), PA (10:0p/22:3), PA (12:0e/20:4), PEt (10:0e/20:4), PEt (8:0p/22:3), PMe (8:0e/23:4), PMe (8:0p/23:3), MGMG (26:3), PA (18:1/18:2), PEt (16:1/18:2), PMe (17:1/18:2), LPA (35:0), LPEt (33:0), LPMe (34:0), PA (16:0e/19:0), PEt (16:0e/17:0), PMe (18:0e/16:0), DG (18:1p/24:7), DG (18:2e/24:7), OAHFA (18:2/27:6), dMePE (13:0/18:3), PE (15:1/18:2), LPA (18:3), MGMG (12:2), PA (16:1/18:2), PEt (10:0/22:3), PMe (15:1/18:2), PS (37:0/18:2), PG (8:0/24:7), DGDG (1:0/18:1), DG (20:1p/24:7), LPA (37:0), LPEt (35:0), LPMe (36:0), OAHFA (18:2/29:6). PA (18:0e/19:0), PEt (16:0e/19:0), PMe (16:0e/20:0), FA (14:0), LPMe (10:0e), CerG1 (d14:0/17:0+O), dMePE (14:0e/16:0), LdMePE (30:0), and LPE (32:0), and PE (16:0e/16.0).

In some embodiments, the lipid sets are derived from vesicles. In some embodiments, the vesicles are microvesicles, exosomes, or a combination thereof. In some embodiments, the microvesicles, exosomes, or combinations thereof are isolated by ultracentrifugation. In other embodiments, the exosomes can be isolated by microfluidics with on chip separation and capture capabilities. However, one of skill in the art will recognize and identify such techniques that enable improved processing and compatibility with clinical laboratory practice. In some instances, a combination of physical separation based on size, with antibody based capture for either exosomes or microvesicles can be used.

In some embodiments, the lipid amounts are determined using mass spectrometry, such as Fourier transform ion cyclotron resonance or Orbitrap mass analyzer. While mass spectrometry is one method of determining lipid amounts, the skilled artisan will readily identify alternative analyses for determining lipid amounts.

The lipid sets are provided in a sample. The samples used are preferably a bodily fluid. In some embodiments the bodily fluid is treated to provide the sample. Treatment can include any suitable method including but not limited to extraction, centrifugation (e.g., ultracentrifugation), lyophilization, fractionation, separation (e.g., using column or gel chromatography), or evaporation. In some instances, this treatment can include one or more extractions with solutions comprising any suitable solvent or combinations of solvents, such as, but not limited to acetonitrile, water, chloroform, methanol, butylated hydroxytoluene, trichloroacetic acid, toluene, hexane, benzene, or combinations thereof. For instance, in some embodiments, fractions from blood are extracted with a mixture comprising methanol and butylated hydroxytoluene. In some instances, the sample (e.g., a bodily fluid extract or a lipid exosome fraction of blood plasma) comprises a concentration of lipid microvesicles that is higher than normally found in a bodily fluid.

In some embodiments, the bodily fluid is selected from the group consisting of blood, urine, nipple aspirate, and BALF. In some embodiments, the bodily fluid originates from breast or lung tissue.

Bodily fluids can be frozen in liquid nitrogen. Preparation of the removed bodily fluids can be performed in any suitable manner.

Some embodiments of the presently-disclosed subject matter provide for a personalized approach to determining a cancer based on the lipid profiles of the subject's neoplasm, including early detection of cancers. For example, in some embodiments, the methods comprise measuring the lipid profile amounts in a control, healthy, or non-cancerous bodily fluid, measuring the lipid profile amounts in a bodily fluid from a subject suspected of having cancer or a condition of interest, comparing the lipid profiles and detecting the increases and decreases of the lipid profiles relative to each other. The increases and decreases of the lipid profiles in a subject suspected of having cancer or a condition of interest relative to the control, healthy or non-cancerous lipid profiles create a pattern in a set of lipids, or lipid set. Such a pattern is analyzed by the predictive models provided herein.

The presently-disclosed subject matter further includes kits comprising a reagent to carry out a method as described herein below.

The terms “treatment” or “treating” refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some embodiments, the non-human subject is a rodent.

While diagnosis typically occurs before treatment, the diagnostic methods described herein, the term “diagnosis” can also mean monitoring of the disease state before, during, or after treatment to determine the progression of the disease state or response to intervention. The monitoring can occur before, during, or after treatment, or combinations thereof, to determine efficacy of therapy, or to predict future episodes of disease.

While the terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

Biomarkers of early stage cancer and methods of early detection, especially in populations at risk, which can be treated by surgery or other early intervention is disclosed herein. The lipid composition of circulating exosomes has been analyzed from the plasma of healthy individuals, those who are accrued to surgical study, and from subjects at all stages of lung cancer. Exosomes and microvesicles were prepared by differential ultracentrifugation as per established protocol. The washed exosomes were solvent extracted and then analyzed by direct infusion high resolution FT-MS. This was performed using a Thermo Fusion Tribrid FT-MS run in both positive and negative modes. Lipid classes were identified by accurate mass using PREMISE, or other means of matching m/z from the FT-MS to a database, and also using tandem MS. Cross-validation of lipids is achieved using the higher resolution SolariX XR FT-ICR-MS, which provides the greatest resolution among commercially-available mass spectrometers, and thus considerably diminished ambiguities.

### Example 1: Breast Cancer and Breast Disease

Lipid profiles of both exosomes and microvesicles were obtained for plasma samples that comprised control, breast cancer and breast disease plasma samples. As shown in Table 1, the samples included healthy, DCIS, infiltrative ductal, infiltrative lobular, other breast cancer and benign samples. Plasma samples received comprised a total of 50 subjects determined by conventional diagnostic methods to be free from breast cancer, 50 patients with invasive lobular or ductal breast cancer, 50 patients with other invasive breast cancers, and 50 patients with ductal carcinoma in situ (DCIS). The disposition of the samples received is summarized in Table 1. Samples were received and analyzed in several batches (Tables 2-4).

TABLE 1
Sample Disposition

| Type | number |
|---|---|
| Healthy | 52 |
| DCIS, | 18 + 2# |
| Infiltrative ductal | 74 + 5# |
| Infiltrative lobular | 8 + 3# |
| Other BrCa | 5# |
| Benign | 5-37* |
| Total infiltrative. | 95 |
| Total Breast Ca | 115-117* |
| Grand total | 204 |

*2 possible BrCa that are actually benign/healthy; # from 15 random mixed cancer (of 15 total); Total analytes ~2.5 million

A first batch of control and breast cancer (BrCa) plasma samples were evaluated, cross-validating previous independent analyses. Mass spectral data collected in positive ion mode were evaluated and the exosomes showed a significant number of potential discriminators. Initial data analysis by OPLSDA showed a clear separation between the control and BrCa cases. The initial training set analysis indicated that there was sufficient power for discriminating exosomal lipid marker patterns of BrCa patients using the positive ion mode FT-MS data, and that 50 each healthy and BrCa samples are needed for the validation set.

A first set of samples received, Table 2, included 105 samples, giving 210 vesicles run by FT-MS in both positive and negative mode yielding a total MS number of runs of 420.

TABLE 2
Samples Analyzed by FT-MS-first group

| | # samples processed and run | |
|---|---|---|
| | on FT-MS | |
| | control | BrCa |
| Exo (+ ion mode) | 52 | 53 |
| Exo (− ion mode) | 52 | 53 |
| MV (+ ion mode) | 52 | 53 |
| MV (− ion mode) | 52 | 53 |
| Total | 208 | 212 |

Exo = exosomes; MV = microvesicles
Total analyses = 420; Total number of analytes ca. 1.26 million

A second batch of 50 samples containing 35 benign breast disease samples was processed to generate exosomes and microvesicles, Table 3. In particular, in Table 3, 35 benign conditions, 1 non-malignant (SBC177), 1 unknown (RPAH 1), and 13 BrCa, of which 10 are invasive carcinomas, were processed.

TABLE 3
Samples Analyzed by FT-MS-second group

| | # samples processed and |
|---|---|
| | run onFT-MS |
| | BrCa |
| Exo (+ ion mode) | 50 |
| Exo (− ion mode) | 50 |
| MV (+ ion mode) | 50 |
| MV (− ion mode) | 50 |
| Total | 200 |

Total analyses = 200; Total number of analytes ca. 0.6 million

A third batch of 50 mixed breast cancer samples was also processed, Table 4. In particular, 18 DCIS, 8 infiltrative lobular, and 24 invasive ductal samples were evaluated.

All of the exosome and microvesicles were analyzed by FT-MS in both positive and negative ion mode as described in the Methods. In summary, the samples were processed into exosomes and microvesicles, and positive and negative ion mode FT-MS spectra were recorded on all exosome samples. OPLS-DA has been carried out as discussed below, and the classification models established.

TABLE 4
Samples Analyzed by FT-MS-third group

| | # samples processed |
|---|---|
| | and run on FT-MS |
| | BrCa |
| Exo (+ ion mode) | 50 |
| Exo (− ion mode) | 50 |
| MV (+ ion mode) | 50 |
| MV (− ion mode) | 50 |
| Total | 200 |

Total analyses = 200; Total analytes ca. 0.6 million

Guided by the OPLSDA results, a statistical classifier model was built using Lasso and ROC for the first 104 samples in positive ion mode has shown that the exosomal lipids discriminate better than the microvesicle lipids.

A second, independent approach to statistical analysis is the Random Forest, not guided by OPLSDA results, which showed excellent separation when a large fraction of all of the FT-MS data is used. The exosomal lipids are statistically superior to the microvesicle lipids, and the AUC for the exosomal lipids (from +ion mode) is also high. The two independent approaches converge on the same conclusions for the first 104 samples, increasing confidence in the analysis.

Comparison of the cancer sets with those from benign disease and with early stage BrCa provided an element of selectivity. The data sets comprise typically >3000 lipid peaks in a single sample, that were assigned to lipid classes (e.g. phosphatidylcholine with C18:1+C16:0, PE, PS, PI, sphingolipids, plasmalogens etc.).

A novel strategy to estimate power and sample size has been developed, which indicates adequate numbers for the training set for discriminating BrCa from healthy, with a power level of >80%. It is also estimated that for validation, 100 blinded, prospective samples are sufficient. Moreover, 35 samples from benign inflammatory breast disease show tight clustering in OPLSDA that separate completely from healthy, and group differently from breast cancer.

Results

Receiver Operator Curve (ROC) analysis of first 100 samples Using a 5 fold cross-validation (20% tests) to construct the ROC, the following was found when analyzing the first 100 samples, Exosomes: AUC=93% with 18 classifiers Microvesicles: AUC=82% with 13 classifiers Creating a model that included both exosomal and microvesicle lipids gave an AUC of about 0.91. This strongly suggests that the information contained within the exosomes is optimal.

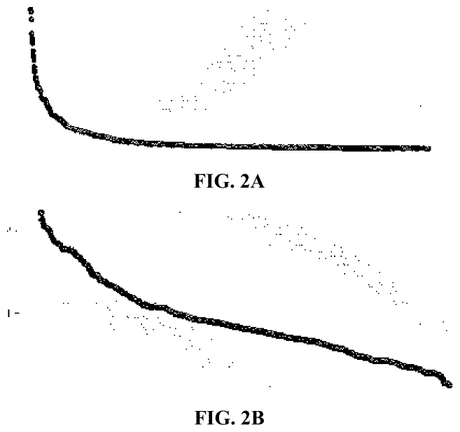

The random forests analysis produced a much sharper cutoff for the lipids discriminators in terms of importance compared with the OPLSDA output (FIG. 2).

Using the importance from FIG. 2, 5-fold cross validation was performed to evaluate lipids to be used as the classifiers. With exosomes, the AUC=0.98 with 7 lipids, with an error rate=9%. In contrast, with microvesicles, the AUC=0.93 with 35 lipids, with an error rate=17%. When combining microvesicles and exosomes, the AUC=0.98 with 14 lipids, with an error rate=8%. Based on this, the combination of exosomes and microvesicles yielded no better results than exosomes alone.

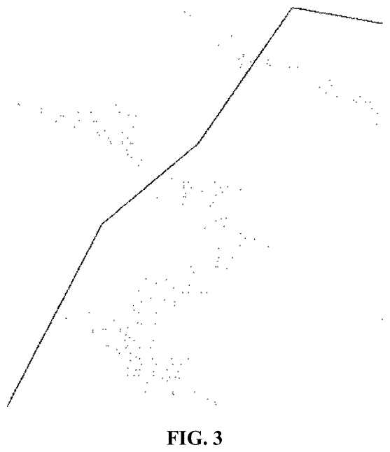

Power analysis was performed analogous to the method described for the Lasso data analysis described herein, with random samples of 12 from each class, and using samples of 20, 25, 30, 35, or 40 from each class for training to obtain AUC (repeated 20 times). FIG. 3 shows that the AUC reached a plateau value of ca. 0.99. These two independent statistical approaches point to the same conclusions, first, that exosomes are better than microvesicles, and second, that combining microvesicles with exosomes produces no benefit. With a current samples size of 104, the AUC is approaching a plateau in the range up to about 0.98. Cross-validation against independent, blinded prospectively collected samples is an important step in evaluation.

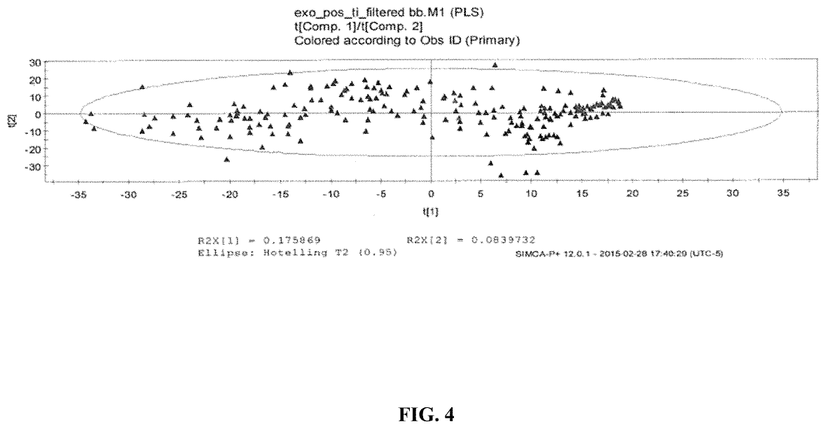

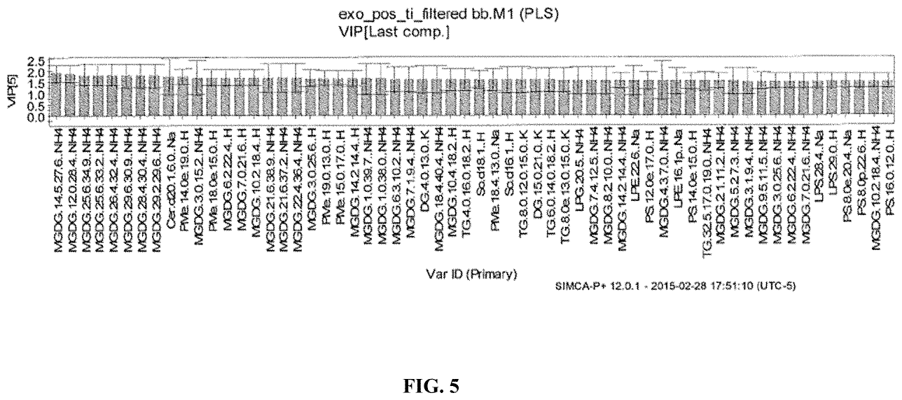

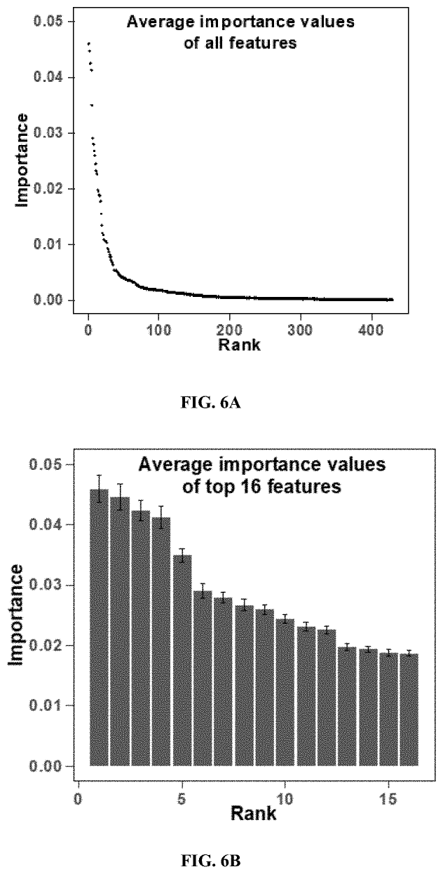

FIG. 4 shows the PCA separation of positive mode exosomes of the three groups (combination of all samples)—healthy, breast cancer and benign breast disease. 204 exosomal lipids in positive ion mode are included, containing 1479 lipids that appear in at least 50% of the samples. The three groups are healthy (N=50); Breast Cancer (N=115); benign breast disease (N=35). Program used—SimcaP+.

For both healthy and BrCa samples, the individuals show wide variance along t1, and partially separate. The variance in t2 is smaller. These groups are largely separated, with some possible outliers. There are two BrCa samples that fell within the healthy group, and two healthy that fell well within the BrCa group. These samples are being checked for possible reasons. Two samples in the BrCa group included one noted as benign, and one noted as unknown diagnosis. The checked data will then be subjected to the Lasso and Random Forest analysis to define the specific and sensitivity.

The 35 designated benign samples (a variety of non-malignant breast conditions) cluster relatively tightly in t1, and overlap only a small fraction of the total range. Clustering relatively tightly in PCA, the samples separated completely from healthy, and partly from BrCa. It is notable too that the cancer samples while having a wide variance, seem also to show a cluster at the right hand side of the PCA.

FIG. 6 shows the left hand side of the SimcaP VIP plot for the evaluated samples. VIP>1.5 is considered significant. This was dominated by features assigned to MGDG (monogalctosyldiacylglycerols), PMe and TG.

Methods

Exosomes and microvesicles were prepared by differential ultracentrifugation as per established protocol. The washed exosomes were solvent extracted and then analyzed by direct infusion high resolution FT-MS, using a Thermo Fusion Tribrid Orbitrap run in both positive and negative ion modes with mass resolution setting of at least 450,000 and mass accuracy of <1 ppm. Lipid classes were identified by accurate mass using in-house software PREMISE, and also by accurate mass plus tandem MS data using Thermo software Lipid Search. Lipid assignment will also be cross-validated using the higher resolution SolariX FT-ICR-MS, which provides a greater mass resolution (up to 10 million), which diminished ambiguities of assignment.

Informatics and Statistics are an integral component of all experimental design, data collection and interpretation. The results of the FT-MS analysis were analyzed by Principal Component Analysis (PCA)/OPLS-DA to demonstrate clean separation between healthy and cancerous samples. These data comprised a training set that can be compared against independent data to be obtained to evaluate the significance. Statistical classifiers are built by Dr. Chi Wang using the Lasso method. Orthogonal machine learning classifiers are built by the Moseley laboratory using the Random Forest method. ROC (Receiver-Operator Characteristics) and power analyses are being carried out by Drs. Chi Wang and Robert Flight on Lasso and Random Forest classifiers, respectively. The ROC is a widely used metric that provided overall accuracy as well as specificity and sensitivity.

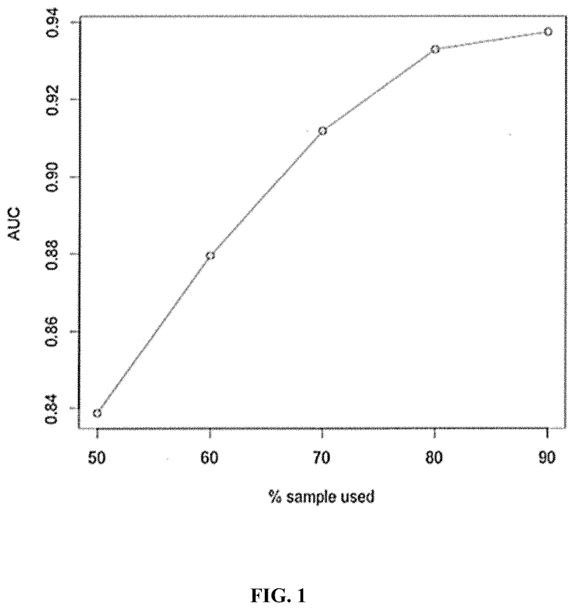

Appropriate classifier from the very rich data sets required several different statistical techniques applied to the problem. The simplest is Orthogonal Partial Least Squares-Discriminant Analysis (OPLS-DA), which was valuable for determining whether there was truly a separation between healthy and lung cancer or other disease states, and provided a list of discriminators to be used to build a classification model. This appeared to be overly generous. Two other approaches, the Random Forest, and the Lasso methods provided clearer discriminator sets, and were used to build classifiers. Satisfyingly, these two different approaches give very similar results, and furthermore, it was possible to develop a novel way to determine the power of the data expressed as a Receiver Operator Characteristics curve (ROC) (FIG. 1), easing design of cross-validation studies with blinded samples. The Area under the ROC Curve (AUC), which is a measure of overall accuracy, was calculated for each model, and plotted against the sample size (taken at random from the total data set). The curve extrapolates to an AUC of ca. 0.96 in this instance, with n-104. Based on the assumption of sampling from an infinite population, the effective power of 80% at a tolerance of 0.05 was achieved for n=34 for these data. The AUC is reaching a plateau by 80% of the total data used (and at infinity reaches 0.96), i.e., good discrimination is possible for such data with <100 data sets, with around 15 discriminators, with both specificity and sensitivity >90%.

The results of the FT-MS analysis were analyzed by PCA/OPLS-DA to demonstrate clean separation between healthy and BrCa data. OPLS-DA provided the main discriminators, from which models were built to find the optimal sensitivity and specificity according to ROC/AUC analysis. These data comprise a training set that can be compared against independent data to be obtained to evaluate the efficacy of lipid patterns of BrCa discerned. An orthogonal approach, Random Forest, was adopted, which finds an optimal solution using all of the data.

Q/C of data. Data analysis began with raw MS data reduction and manual curation, followed by PCA and OPLS-DA, useful tools for initial visualization of the data. PCA showed whether groups separate, and OPLSDA provided some information about the discriminators if there was group separation.

Furthermore, it was possible to identify individual samples that appeared as outliers. These outliers may be due to inaccurate assignments in the data reduction stage, to a poor sample preparation or to a bad MS run. Outliers were then re-analyzed (second phase of manual curation) to establish whether the MS run was bad. If not, then it may be the sample, which must then be reprocessed and re-run. If this was not the reason for the outlier, then the code must be broken to determine whether there was a factor in the individual that might explain the outlier behavior (e.g. misassignment of group-healthy/cancer/benign).

Positive Ion Mode Signals Statistical Analysis. The following analysis represents an independent cohort, with samples from different sites, processed by different operators, analyzed on different instrumentation using different software. In what follows, the statistical analyses refer to positive ion mode signals only, and are done by binary comparison of BrCa against healthy cohorts.

Receiver Operator Curve (ROC) analysis of first 100 samples. The Lasso method was used to determine the discriminators from the lipid data of exosomes and microvesicles separately. This method found the best linear combination of lipid species (feature) to predict the outcome according to the simple model

$$log \frac{P ( an individual is a case )}{P ( an individual is a control )} = β \text{?} + β \text{?} x \text{?} + … + β \text{?} x \text{?}$$

$$\text{?} \text{indicates text missing or illegible when filed}$$

where xₖ, k=1, . . . , K, is the expression value of feature k for that individual. The beta parameters are the weights for each feature, many of which are small and do not contribute. These features are filtered out according to an inverse linear sliding scale.

Power and sample size: The current techniques for estimating power and samples size were not well suited to data of this kind. The question was formulated to determine the number of samples that are needed to build the classifier set, and the number of sample needed to estimate accuracy of the AUC with good confidence intervals. For the model building, it was assumed that features are 0 normally distributed and independent. With these <assumptions, the standardized fold change=log(fold change)/sd=1.62 for the exosomes, and only 1.01 for the microvesicles. The sample size is the actual number of samples needed to produce a correct classifier referenced to the infinite population size. If one accepts a tolerance of 0.05 (analogous to a p value of 0.05) for the difference between the best prediction and that for an infinite data set, then n=34 (17 each for controls and case) for the exosomes, and 74 for the microvesicles.

The training set had n=104. As this is nearly 3× the size estimated, it may be robust even for significant deviation from normality. Randomly chosen subsamples from the n=104 exosome data were used as training sets, and the remainder as validation sets; this process was repeated many times. FIG. 1 shows a graphical representation of how the AUC varies as a function of sample size. Clearly the AUC is reaching a plateau value above n=80. To reach 80% power, n=72 is needed to achieve a CI of >0.8. To improve the CI to 0.865 approximately twice as many samples would be needed. At least 100 samples for the validation set could help achieve the targeted CI.

Random Forests: Random forests are an ensemble learning method for classification that operate by constructing a large number of decision trees with random sampling. Bootstrap: sampling with replacement (control error). Aggregation: lots of trees. Each point in each tree uses a random subset of lipids.

Exosome Isolation: Alternatives to Ultracentrifugation (U/C)

The current “gold standard” for exosome isolation is differential ultracentrifugation. While relatively slow (presently about 1 h per sample), it does produce a homogeneous distribution of particles and a separate distribution of microvesicles, without any selection other than buoyant mass and size. Other isolation kits are faster, but rely primarily on size and do not give both exosomes and microparticles. They give rise to less homogeneous preparations. Antibody-based selection selects only those particles bearing a specific antigen, and does not fit the operational definition of exosomes.

Microfluidics with on chip separation and capture capabilities offer the possibility of replacing ultracentrifugation with a fast process, which is better compatible with clinical laboratory practice. Several such approaches have been described, using a combination of physical separation based on size, with antibody based capture for either exosomes or microvesicles. Conceivably these could be combined in a single device. A microfluidic device has been designed that uses antibody capture of the exosomes. A microfluidic device has been received for testing rapid isolation of exosomes from blood plasma. Selectivity will be assessed by analyzing data from benign breast disease and an independent set of lung cancer samples.

Another option is using a Malvern Nanosight instrument (Malvern Instruments Ltd.), which directly counts the particles, as well as providing their size (www.malvern.com).

There are advantages and disadvantages of UC plus MS analysis compared with alternative methods. It appears that a MF-based separation method is rapid and could work with plasma or even whole blood, by separation according to size. Absolute counting is unlikely to be reliable, considering dilution by impurities. Antibody capture requires broad selectivity for exosome or MVs that are specific for the disease state. Literature suggests that there may be different antigen sets according to the disease states, making a capture system based on antibodies more complex. Antibody based detection requires different and highly specific sets of antibodies, which can be multiplexed.

FT-MS has the advantage of multiple disease diagnosis with different sets of discriminators, such as cancer versus non cancer versus inflammatory disease, versus liver damage (etc.). It is also feasible to discriminate between types of cancer. Once profiles specific for different diseases have been validated, simpler detection methods are plausible. FT-MS is not standard for clinical labs. Specialized diagnostic labs however could be established to provide such service. FT-MS is very fast (<10 min measurement) and subsequent analysis using informatics tools for discerning specific signatures in the very large data set can be automated.

Utilization of combined exosome isolation kits with U/C is one way to evaluate the exosome signatures. There are several exosome isolation kits on the market that utilize polymers to trap exosomes, although they tend to give impure preparations for FT-MS analysis. Preliminary tests performed on some of these kits (e.g. PureExo) shows that in combination with a single U/C step, exosomes are purified and suitable for FT-MS analysis. However, MV are lost with this method.

The kits can process samples in parallel. A single ultracentrifuge run of 1 h plus samples handling takes about 2 h. The whole process with manual operation would take about 4-5 h, for processing 6 samples simultaneously. With robotic samples handling this might be reduced to ca 2 h/6 samples (i.e. 20 minute per sample).

### Discussion

Breast cancer and control samples are well separated using exosomal lipids as discriminators (Classification). OPLS-DA shows that the BrCa samples separate cleanly from the control samples with each data point representing 1200 assigned lipids in the positive ion mode (out of 3060 detected ions). These amounted to a total of >1.2 million lipid species analyzed. With tandem MS, some limited differentiation of the fatty acyl chain is possible, based on the mass loss, but there remains ambiguity, thus specific identification was sought once the statistical model of the lipid discriminators was established. In statistical analyses of positive ion mode signals, exosomes showed the ability to outperform microvesicles in positive ion mode. Moreover, 35 samples from benign inflammatory breast disease show tight clustering in OPLSDA that separate completely from healthy, and also group differently from breast cancer.

### Example 2: Lung Cancer

Blood samples have been obtained from more than 80 healthy individuals, and blood samples acquired not only from the surgical cohort (presently >200 samples) but also blood samples from individuals with more advanced stage cancers, including advanced stage NSCLC, with and without chemotherapy from an oncology clinic. Using FT-MS, we can resolve 3000 features in each lipid extract of the exosomes. The data collection time for each lipid extract was 5-10 minutes. However, automated data reduction will speed up the data analysis. Early stage NSCLC lipids separated well from healthy controls, and also from breast cancer. An important control will be to determine whether the lung cancer and inflammatory lung disease also separates, as then this provides a tool for screening populations at risk, with potentially a much lower false positive rate than the current SOC, spiral CT.

TABLE 5
Lipid Classifiers

| Class | | Total | # |
|---|---|---|---|
| TG | 1 | 68:5; 68:6; 22:6; 51:0; 67:6; 71:6; | 17 |
| | | 77:6; 46:4; 58:6; 56:6; 75:6; 52:2; | |
| | | 50:0; 42:1; 43:2; 34:2; 35:2 | |
| DG | 2 | 24:2; 38:6; 53:6; 17:0; 21:0; 28:0; | 8 |
| | | 40:8; 38:8 | |
| PIP2 | 3 | 42:7; 48:7; 46:7; 41:0 | 4 |
| PIP | 4 | 55:6; 29:3; 29:2; 30:6; 48:8; 46:5; | 6 |
| MGDG | 5 | 23:6; 45:10; 46:10; 42:6; 27:7; 37:8; | 11 |
| | | 26:1; 27:1; 7:0; 33:15; 13:6 | |
| MGMG | 6 | 23:10; 11:3 | 2 |
| MG | 7 | 14:0; 18:0 | 2 |
| PC | 8 | 34:7; 33:0; 32:0; 34:6; 28:0; 28:3; | 8 |
| | | 25:0; 28:2 | |
| PS | 9 | 23:0; 37:2 | 2 |
| PE | 10 | 29:0; 31:2; 31:3; 30:8; 30:3; 28:0 | 6 |
| PG | 11 | 32:0; 37:4 | 2 |
| dMePE | 12 | 28:1; 8:0; 29:3; 29:2; 28:2; 28:3; 26:0 | 7 |
| So | 13 | d16:1 | 1 |
| LPG | 14 | 12:0; 15:0 | 2 |
| LdMePE | 15 | 27:0; 28:3; 27:4; 29:3; 26:0; 28:4 | 3 |
| LPC | 16 | 26:0; 25:0; 27:3; 28:3 | 4 |
| LPE | 17 | 29:0; 28:0; 30:3; 8:0 | 4 |
| LPI | 18 | 16:1 | 1 |
| Pet | 19 | 28:2; 31:2; 22:2 | 3 |
| CerG2GNAc1 | 20 | 34:2; | 1 |
| Cer | 21 | 24:1; 26:0; 24:0; | 3 |
| LPA | 22 | 33:4; 32:4 | 3 |
| PA | 23 | 23:4; 33:3; 32:3; 32:4; 33:2; | 6 |
| | | 24:2; 32:2 | |
| PI | 24 | 51:8 | 1 |
| cPA | 25 | 18:2; 16:0 | 2 |
| LPEt | 26 | 30:4 | 1 |
| phSM | 27 | 27:4; 27:1; 28:1; 28:0; 28:4; | 5 |
| PMe | 28 | 31:23; 32:2; | 2 |
| 28 | | | 117 |

Abbreviations used in Table 5 are as follows: TG—triacylglycerol, DG—diacylglycerol, PIP2—phosphatidyl inositol bisphosphate, PIP—phosphatidyl inositol phosphate, MGDG—Monogalactosyldiacylglycerol, MGMG—monogalactosylmonoacylglycerol, MG—monoacylglycerol, PC—phosphatidyl choline, PS—phosphatidyl serine, PE—phosphatidyl ethanolamine, PG—phosphatidyl glycerol, dMePE—dimethylphosphatidyl ethanolamine, So—Sphingosine, LPG—lyso phosphatidyl glycerol, LdMePE—lyso dimethylphosphatidyl ethanolamine, LPC—lyso phosphatidyl choline, LPE—lyso phosphatidyl ethanolamine, LPI—lyso phosphatidyl inositol, Pet—phosphoethanolamine, Cer—ceramide, CerG2GNAc1—neutral glycosphingolipid, LPA—lyso phosphatidic acid, PA—phosphatidic acid, PI—phosphatidyl inositol, cPA—cyclic phosphatidyl acid, LPEt—lysophosphoethanoloamine, phSM—phosphosphingomyelin, and PMe—phosphomethanol.

Early detection of non-small cell lung cancers: Investigation of the lipid profiles of blood plasma exosomes using ultra high-resolution Fourier transform mass spectrometry (UHR-FTMS) for early detection of the prevalent non-small cell lung cancers (NSCLC) was conducted. Plasma exosomal lipid profiles were acquired from 39 normal and 9 NSCLC subjects (44 early stage and 47 late stage). Two multivariate statistical methods, Random Forest (RF) and Least Absolute Shrinkage and Selection Operator (LASSO) have been applied to classify the data. For the RF method, the Gini importance of the assigned lipids was calculated to select 16 lipids with top importance. Using the LASSO method, 7 features were selected based on a grouped LASSO penalty. The Area Under the Receiver Operating Characteristic curve for early and late stage cancer versus normal subjects using the selected lipid features was 0.85 and 0.88 for RF and 0.79 and 0.77 for LASSO, respectively. These results show the value of RF and LASSO for metabolomics data-based biomarker development, which provide robust and orthogonal classifiers with sparse data sets.

Exosomes and Microvesicles Carry Tumor Cell-Derived Bioactive Materials.

Interestingly, both SM and PS have been linked to lipid microparticles (MP) shed from cells. MP such as exosomes (EXO) and microvesicles (MV) can be shed from many different cell types, most notably immune cells and tumor cells, into the circulating blood. EXO are multivesicular bodies originating from the endosomal membrane, and are released upon fusion with the plasma membrane while MV are formed by outward budding and fission of the plasma membrane. Both types of lipidic MP are thought to mediate extracellular communications such as immune activation or suppression. MP derived from cancer cells including lung cancer cells can carry a variety of bioactive proteins (e.g. epidermal growth factor receptor, EGFR; vascular endothelial growth factor, VEGF; integrins; Fas ligand; latent membrane protein, LMP-1; angiogenic factor tetraspanin; macrophage migration inhibitory factor or MIF) and microRNAs to promote tumor growth/invasion/metastasis as well as to enact immune evasion and drug resistance. Although largely unexplored, exosomal lipids derived from cancer cells have been shown to elicit apoptosis in sensitive cells via inhibition of the Notch-1 pathway but activate the Akt survival pathway via promoting the NFκB-SDF1-CXCR4 axis in resistant cells. Melanoma cells cultured under acidic conditions released EXO with a higher SM content, and were shown to have a higher capacity for cell fusion and delivery of caveolin-1 (tumor promoting) to less aggressive melanoma cells than neutral EXO. Moreover, blocking CE buildup interferes with exosomal uptake and has anti-cancer effects, while ceramide buildup is important for exosomal biogenesis and triggers cancer cell death. Thus, there are vital functions of lipids in exosomal biogenesis and interactions with the tumor microenvironment (TME) to influence tumor development and progression.

Recently, exosomal components such as microRNA and proteins have been shown to be promising diagnostic tools in human cancers including lung cancer. However, it is unclear if these components can be generally useful in classifying lung cancer, as the microRNA signatures did not differ qualitatively between lung cancer and normal subjects while the accuracy of protein markers for advanced stage NSCLC detection was only 75%. Such limitations do not meet the specificity and sensitivity requirements for lung cancer screening at early stages.

We have procured blood plasma samples from 39 normal and 91 NSCLC subjects (44 early stage and 47 late stage) for EXO isolation and lipid profiling using UHR-FTMS. We also applied two advanced multivariate statistical methods, Random Forest (RF) and Least Absolute Shrinkage and Selection Operator (LASSO) to perform supervised clustering analysis of the EXO lipid profiles. The Area Under the Receiver Operating Characteristic curve (AUROC) of normal versus early and late stage NSCLC using the top 16 (for RF) or top 7 (for LASSO) lipid features was 0.85 and 0.88 or 0.79 and 0.77, respectively. These results showed that selected lipid species of plasma EXO discriminated normal from early and late stage NSCLC and demonstrate the value of RF and LASSO for metabolomic data-based biomarker development.

Material and Methods

Blood Collection A total of 131 blood samples were collected prospectively with informed consent under University of Kentucky IRB-approved protocols from 39 normal volunteers, 44 patients undergoing surgery for early stage (I, II) lung cancer and 47 patients with advanced NSCLC (stages III, IV) attending the multiD clinic. The age range was 40-85 y and there were similar number of males and females, and overall the population was >95% Caucasian.

Approximately ten mL samples of blood were drawn into a purple top vacutainer containing K₂-EDTA (Becton-Dickson), inverted twice to ensure dissolution of the EDTA, and kept on ice immediately after blood draw. The whole blood was separated into packed red cells, buffy coat, and plasma within 30 minutes of collection by centrifuging at 3,500 g for 15 min at 4° C. in a swing out rotor. Wherever possible, all blood processing procedures were performed in a class II biosafety cabinet housed in a BSL category 2 laboratory. Plasma (0.7 mL) was aliquoted into 1.5 mL screw cap vials, flash frozen in liq. N₂, and stored at −80° C. until exosomal isolation. These collection and processing procedures were designed to minimize variations in plasma and exosome quality.

Exosome preparation. Exosomes were isolated from plasma by ultracentrifugation. 0.7 mL plasma were placed in 1 ml polyallomer ultracentrifuge tubes on ice, and centrifuged for 1 h at 70,000 rpm at 4° C. in a SWTi55 swing out rotor (Beckman). The supernatant was recentrifuged at 100,000 g for 1 h at 4° C., and the pellet was drained and resuspended in 0.7 mL cold PBS, and recentrifuged at 100,000 g for 1 h at 4° C. The washed exosomal pellets were resuspended in 100 μL nanopure water, vortexed for 30 sec and transferred to a fresh microcentrifuge tube. The ultracentrifuge tube was washed with another 100 μL of nanopure water, vortex for 30 sec and the wash was transferred into same microcentrifuge tube, using the same pipet tip. The combined exosome suspensions were then lyophilized.

The lyophilized EXO preparations were extracted for lipidic metabolites using a solvent partitioning method with CH₃CN:H₂O:CHCl₃ (2:1.5:1, v/v) as described previously. The resulting lipid extracts were vacuum-dried in a vacuum centrifuge (Eppendorf), redissolved in 200 μL CHCl₃:CH₃OH:butylated hydroxytoluene (2:1:1 mM) and diluted 1:20 in isopropanol/CH₃OH/CHCl₃/ammonium formate (4:2:1:20 mM) before analysis for lipids using our ultra high-resolution FTMS (see below) at a resolving power of >400,000 with sub ppm mass accuracy at m/z of 400.

Methods

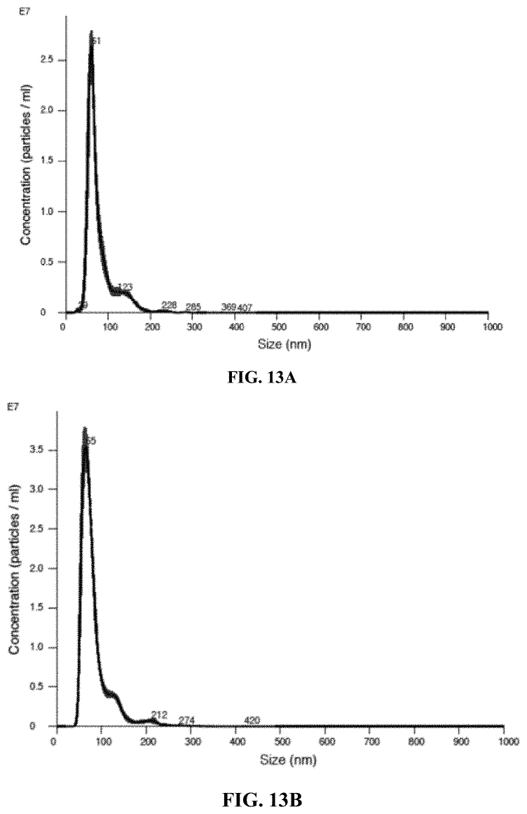

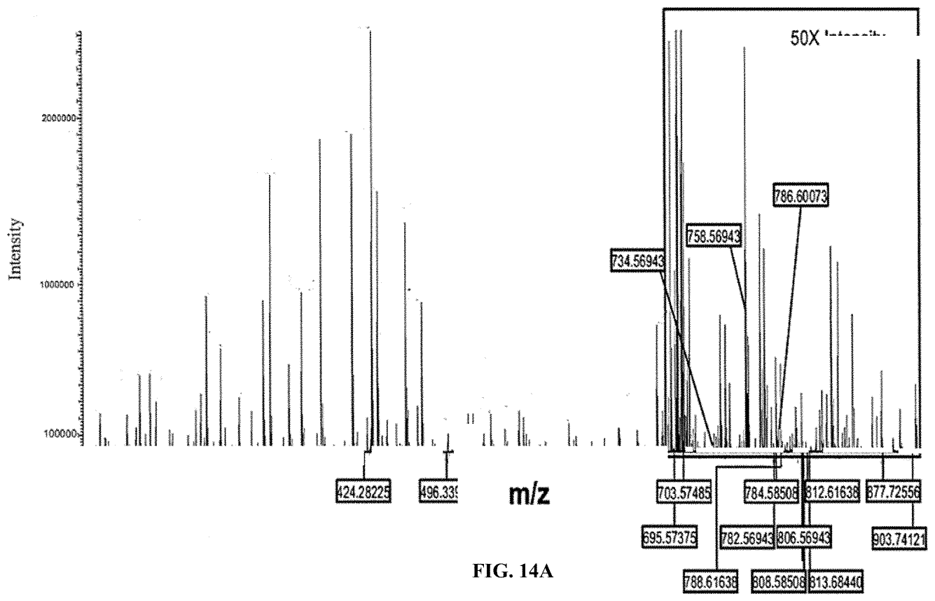

Microparticle characterization A small fraction (<1%) of each exosome preparation was characterized by size distribution analysis using a Nanosight 300 (Malvern Instruments), which provided the distribution of the Stokes' radius (mean 60-66 nm) and the number density of the particles. A typical analysis is shown in FIG. 13. The method eliminates very small particles, and provides a strongly peaked, narrow distribution at the expected size for exosomes (40-100 nm, observed mode of 60-65 nm for the main peaks in FIG. 13A,B).

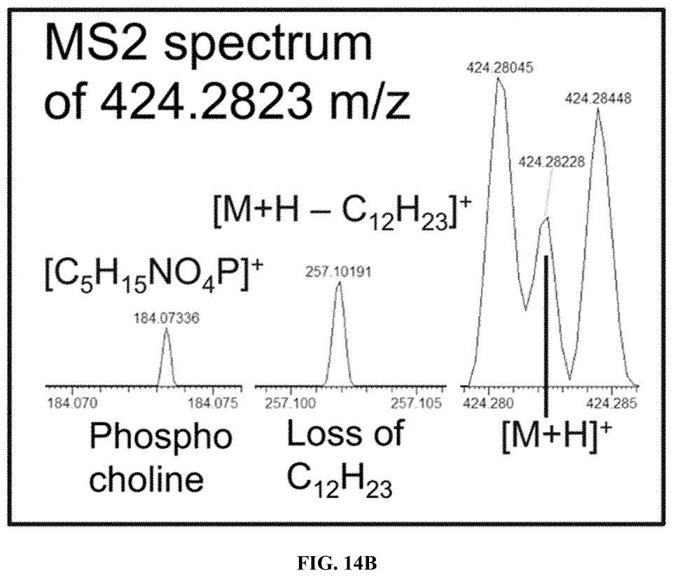

UHR-FT-MS analysis of exosomal lipids High sample throughput (≤16 min total cycle time per sample, <7 min for MS1 portion) was achieved using the nanoelectrospray TriVersa NanoMate (Advion Biosciences, Ithaca, N.Y., USA) with 1.5 kV electrospray voltage and 0.4 psi head pressure. UHR-FTMS data were acquired from an Orbitrap Fusion Tribrid (Thermo Scientific, San Jose, Calif., USA) set at a resolving power of 450,000 (at 200 m/z) for MS1 full scans using 10 microscans per scan in the m/z range of 150-1,600, achieving sub ppm mass accuracy through >800 m/z in positive mode. AGC (Automatic Gain Control) target was set to 1e5 and maximal injection time was set to 100 ms. During the MS1 run, the top 500 most intense monoisotopic precursor ions were isolated via quadrupole using 1 m/z isolation window and HCD (Higher Energy Collisional Dissociation) set at 25% collision energy was performed in positive mode for data-dependent MS2 at a resolving power of 120,000 (at 200 m/z) to obtain fragments for acyl chain assignment and neutral loss of specific head groups. The AGC target was set to 5e4 with maximal injection time of 500 ms. MS2 does not distinguish the sn1 and sn2 acyl positions of glycerolipids, nor the position of unsaturations in acyl chains and acyl branching. Representative full scan MS along with an example MS2 spectrum are shown in FIG. 14.

The UHRMS raw data were assigned by our (CESB) in-house software PREMISE (PRecalculated Exact Mass Isotopologue Search Engine) that compares UHR-FTMS m/z data against our metabolite m/z library (calculated with mass accuracy to the 5th decimal point) to discern all known lipids and their 13C isotopologues, including hypothetical lipids, while simultaneously taking into account all of the major adducts (here H+, Na+, K+ and NH4+). An in-house developed natural abundance (NA) correction algorithm was applied to simultaneously examine the distribution of naturally occurring 13C isotopologues of the unlabeled lipids to help verify the assigned molecular formulae, and to eliminate non-monoisotopic 13C isotopologues from further analysis. For statistical classification, we used only high accuracy monoisotopic m/z values that mapped to lipid molecular formulae, and multiple adducts of each were tracked throughout to avoid redundancy. Below, such m/z values are referred to as “lipid features”, and neither molecular formulae nor lipid names were directly used.

The number of assigned lipid features in each sample varied from 1 to 70. After combining all samples into a master file, the data set had a total of 430 such lipid features. Prior to multivariate statistical analyses, MS1 peaks arising from solvent blanks and known contaminants were removed from the lipid feature lists. As absolute intensities vary from sample to sample, the lipid features must be normalized. The intensities of the lipid features in each sample were thus normalized to the summed intensities of all mass peaks that were non-zero in 20%, 50%, 75%, 97%, 100% of all samples. This is equivalent to estimating the mole fraction of each lipid feature present, and therefore can be used for determining relative changes in composition. We found that normalization using the summed intensities of lipid features that were non-zero in 20% of all samples provided the best statistical outcome according to the ROC analysis.

Multivariate Statistical Analyses

Principal Component Analysis (PCA) and Orthogonal Partial Least Square Discriminant Analysis (OPLS-DA) PCA and supervised OPLS-DA were performed using the SIMCA-P software package (Umetrics, Umea Sweden) to visualize group separation and data outliers although no outliers were removed from the RF and LASSO analysis. PCA model with two components and OPLS-DA model with one predictive component were built. The explained variation (R2) of each component in PCA and OPLS-DA was reported.

Random Forest (RF) Random forest is a supervised classifier developed by Breiman that assembles prediction results of a number of classification and regression trees (CART). Bootstrap sampling was used in the CARTs with random training sampling and replacement to fit each tree. The prediction results were calculated by averaging the results of all trained tree predictors. Bootstrap sampling and ensemble methods provided superior performance for RF analysis. Besides classification, RF provided the importance of the lipid features based on the Gini impurity reduction in every tree. Pairwise proximity between samples was also calculated according to the frequencies of splitting to the same nodes in the forest trees. This helped visualize the dataset clustering status and detect outliers. The RF classification analysis was performed using scikit-learn (version 0.18rc2) library in Python (version 2.7.13). The proximity analysis was performed with the Random Forest package (version 4.6-12) in R (version 3.3.1)

Lasso In parallel to RF analysis, we performed the LASSO regression analysis on the same datasets. Specifically, a multinomial regression model was implemented to classify subjects into normal, early stage lung cancer, or late stage lung cancer groups, where a predicted probability for an individual belonging to each of the three groups was obtained from the model. A grouped lasso penalty was used for feature selection, which ensured that the multinomial coefficients for a variable were all in or out together in the model. The analysis was performed based on the glmnet package (version 2.0-5) in R (version 3.3.1).

Classification performance evaluation For both methods, the classification performance was evaluated by 5-fold cross validation, where four fifths of the data were used for feature selection and model construction, and the area under the Receiver Operation Characteristic curve (AUROC), sensitivity and specificity of the model were evaluated based on the hold-off one fifth data. After each round of classification test, the exact mass list chosen by the RF or LASSO analysis as lipid features was examined and removed if they overlapped with noise, contaminant, or other artifactual peaks. Classification tests and artifact removal were preformed iteratively until the selected lipid feature list contained no known artifacts. For the final RF and LASSO classification test, the top 16 and 7 features were selected, respectively. For RF, the 5-fold cross validation was replicated 500 times. The average AUROC, sensitivity, specificity as well as their 95% confidence intervals were reported. See also additional information and data in Fan, T. W M.*, Zhang, X., Wang, C., Yang, Y., Kang, W Y., Higashi, R. M., Liu, J. & Lane, A. N.* (2018) Exosomal lipids for classifying early and late stage non-small cell lung cancer. Anal. Chim. Acta Sp. Iss. Accepted for publication.

Results

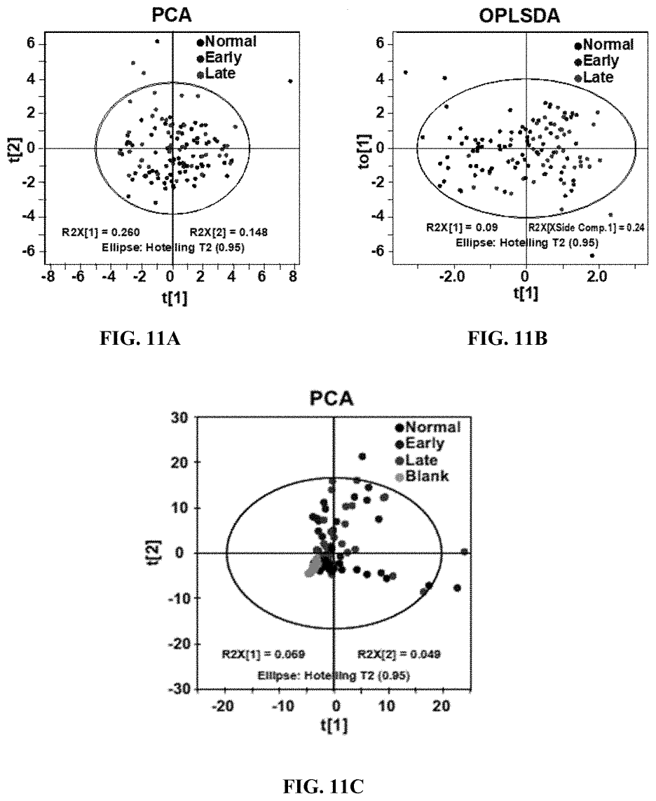

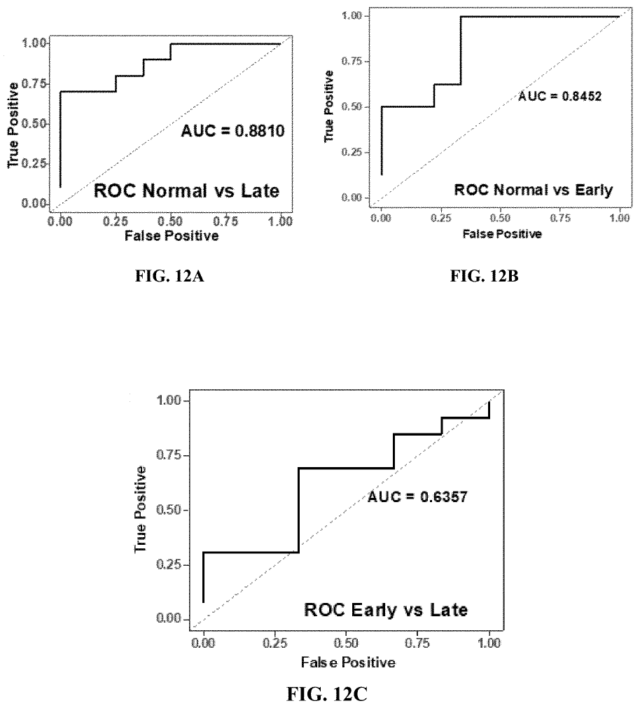

Exploratory analysis with PCA and OPLSDA First, the normalized and blank-removed exosomal lipid data was analyzed using classical unsupervised PCA and supervised OPLSDA methods to visualize data outliers. As shown in FIG. 11, only a few outliers were evident in both types of analysis. We also noted that the PCA method did not yield a clear separation of normal from the early or late stage lung cancer subjects (FIG. 11A), while the separation with the OPLSDA method was somewhat better (FIG. 11B), although this supervised method tended to overfit models to data.

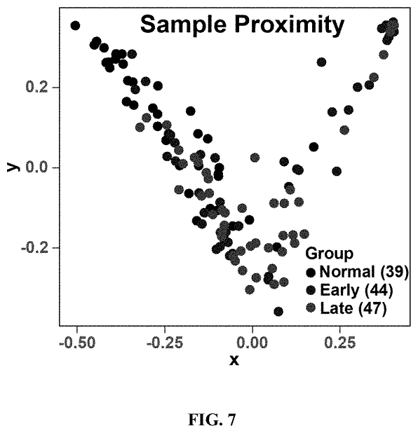

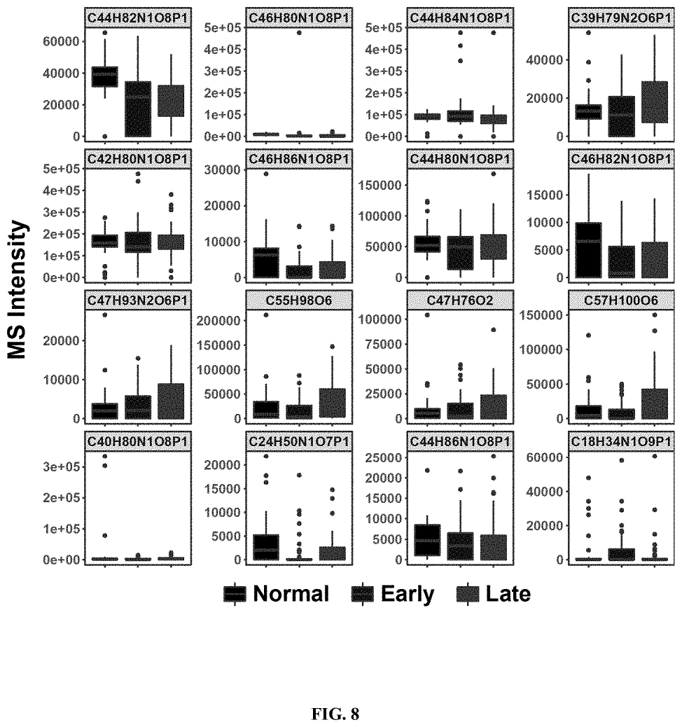

Classification performance of Random Forest The Gini importance of a total 430 lipid features was calculated using the RF method. The number of decision trees was set to 500 based on the results of parameter tuning tests. The importance status is shown in FIG. 6A. Based on the 500 decision tree test, about ⅔ of the 430 features had importance value equal or close to 0. This showed that only ⅓ of the assigned lipid features had the capacity to discriminate different lung cancer stages. The 16 lipid features with highest Gini importance (FIG. 6B) were selected for classification. The classification results of normal versus early and late lung cancer as well as early versus late lung cancer are shown in Table 6 and FIG. 12. The calculated AUROCs for the normal versus cancer were ≥0.85 with low standard deviations, which shows the promise of using the exosomal lipid features for classifying lung cancer. In contrast, the AUROC of early versus late stage cancer was lower (0.64), which suggests a lower potential for exosomal lipid features as classifiers of different stages of lung cancer. The AUROC results were consistent with the RF proximity plot (FIG. 7), which showed good clustering of normal versus cancer with few outliers but not early versus late stage cancer.

The distribution of the MS peak intensity of the top 16 lipid features (shown as molecular formulae) for classifying the three subject groups is shown as boxplots in FIG. 8. They illustrated both positive and negative changes from normal to early and late stage lung cancer. These 16 lipid features were confirmed for their identity based on both accurate mass and MS² fragmentation patterns, as shown in Table 7. Many of the top lipid features were phosphatidylcholines (PC) containing polyunsaturated fatty acyl (PUFA) chains, two were SM known to be enriched in exosomes, and two were lysophosphatidylcholines (LPC) shown to promote exosome biogenesis and lymphocyte chemotaxis.

Classification performance of Random Forest The Gini importance of a total 430 lipid features was calculated using the RF method. The number of decision trees was set to 500 based on the results of parameter tuning tests. The importance status is shown in FIG. 6A. Based on the 500 decision tree test, about ⅔ of the 430 features had importance value equal or close to 0. This showed that only ⅓ of the assigned lipid features had the capacity to discriminate different lung cancer stages. The 16 lipid features with highest Gini importance (FIG. 6B) were selected for classification. The classification results of normal versus early and late lung cancer as well as early versus late lung cancer are shown in Table 6 and FIG. 12. The calculated AUROCs for the normal versus cancer were ≥0.85 with low standard deviations, which shows the promise of using the exosomal lipid features for classifying lung cancer. In contrast, the AUROC of early versus late stage cancer was lower (0.64), which suggests a lower potential for exosomal lipid features as classifiers of different stages of lung cancer. The AUROC results were consistent with the RF proximity plot (FIG. 7), which showed good clustering of normal versus cancer with few outliers but not early versus late stage cancer.

The distribution of the MS peak intensity of the top 16 lipid features (shown as molecular formulae) for classifying the three subject groups is shown as boxplots in FIG. 8. They illustrated both positive and negative changes from normal to early and late stage lung cancer. These 16 lipid features were confirmed for their identity based on both accurate mass and MS² fragmentation patterns, as shown in Table 7. Many of the top lipid features were phosphatidylcholines (PC) containing polyunsaturated fatty acyl (PUFA) chains, two were SM known to be enriched in exosomes, and two were lysophosphatidylcholines (LPC) shown to promote exosome biogenesis and lymphocyte chemotaxis.

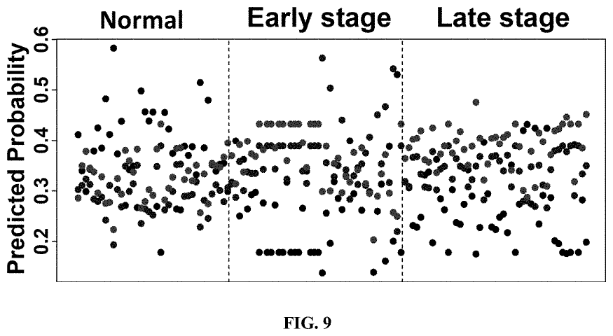

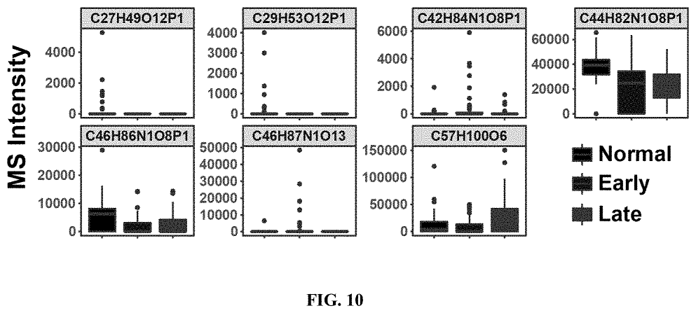

Classification performance of LASSO The LASSO method selected 7 out of the 430 lipid features to construct a multinomial regression model. FIG. 9 showed the model-predicted probabilities for each subject to be in each of the three disease status groups. For many patients, the predicted probability of belonging to the true disease group was the highest, indicating that the model was able to accurately classify a large fraction of the subjects. The MS intensity distributions of the 7 features in the three subject groups were plotted in FIG. 10. To more rigorously evaluate the performance of LASSO, a 5-fold cross validation was performed as described in the Methods section. The AUROCs for discriminating normal versus early and late stage lung cancer were 0.79 and 0.77, respectively (Table 6), which was somewhat lower than those for the RF method.

TABLE 6
Exosomal lipid-based classification of normal versus early and late stage NSCLC using RF and LASSO.

| | | | | | | | | | |
|---|---|---|---|---|---|---|---|---|---|
| Subjects | AUROC | Std | 95% CI | Sensitivity | Std | 95% CI | Specificity | Std | 95% CI |

Random Forest (with top 16 features)

| | | | | | | | | | | | | |
|---|---|---|---|---|---|---|---|---|---|---|---|---|
| Normal | 0.85 | 0.09 | 0.62 | 0.99 | 0.77 | 0.16 | 0.43 | 1.00 | 0.72 | 0.17 | 0.38 | 1.00 |
| vs_Early | | | | | | | | | | | | |
| Normal | 0.88 | 0.08 | 0.69 | 1.00 | 0.84 | 0.12 | 0.57 | 1.00 | 0.72 | 0.16 | 0.42 | 1.00 |
| vs Late | | | | | | | | | | | | |
| Early vs | 0.64 | 0.12 | 0.41 | 0.84 | 0.67 | 0.16 | 0.31 | 1.00 | 0.54 | 0.17 | 7 0.22 | 0.89 |
| Late | | | | | | | | | | | | |

LASSO (with top 7 features)

| | | | | | | | | | | | | |
|---|---|---|---|---|---|---|---|---|---|---|---|---|
| Normal | 0.79 | 0.04 | 0.71 | 0.85 | 0.65 | 0.09 | 0.46 | 0.78 | 0.77 | 0.05 | 0.66 | 0.85 |
| vs_Early | | | | | | | | | | | | |
| Normal | 0.77 | 0.04 | 0.68 | 0.83 | 0.54 | 0.08 | 0.37 | 0.70 | 0.82 | 0.04 | 0.73 | 0.89 |
| vs Late | | | | | | | | | | | | |
| Early vs | 0.51 | 0.05 | 0.41 | 0.61 | 0.33 | 0.09 | 0.16 | 0.51 | 0.73 | 0.09 | 0.53 | 0.90 |
| Late | | | | | | | | | | | | |

However, the LASSO method gave higher specificity indices than the RF method (Table 6). We were able to confirm the lipid identity on 3 out of the 7 lipid features, which overlapped with those revealed by the RF method (Table 7).

TABLE 7
Exosomal lipid-based classification of normal versus early and late stage
NSCLC using RF and LASSO.

| | | accurate | | | |
|---|---|---|---|---|---|
| MF¹ | adduct | mass² | lipid assignment³ | RF | LASSO |
| C44H82N1O8P1 | [M + H]⁺ | 784.58508 | PC(18:1_18:2) | Yes | Yes |
| C44H84N1O8P1 | [M + H]⁺ | 786.60073 | PC(18:0_18:2) | Yes | No |
| C46H80N1O8P1 | [M + H]⁺ | 806.56943 | PC(16:0_22:6) | Yes | No |
| C42H80N1O8P1 | [M + H]⁺ | 758.56943 | PC(16:0_18:2) | Yes | No |
| C39H79N2O6P1 | [M + H]⁺ | 703.57485 | SM(d18:1_16:0) | Yes | No |
| C46H86N1O8P1 | [M + H]⁺ | 812.61638 | PC(18:0_20:3) | Yes | Yes |
| C44H80N1O8P1 | [M + H]⁺ | 782.56943 | PC(16:0_20:4) | Yes | No |
| C46H82N1O8P1 | [M + H]⁺ | 808.58508 | PC(16:0_22:5) | Yes | No |
| C47H76O2 | [M + Na]⁺ | 695.57375 | CE(20:4) | Yes | No |
| C55H98O6 | [M + Na]⁺ | 877.72556 | TAG(52:5) | Yes | No |
| C47H93N2O6P1 | [M + H]⁺ | 813.68440 | SM(d18:1_24:1) | Yes | No |
| C44H86N1O8P1 | [M + H]⁺ | 788.61638 | PC(18:0_18:1) | Yes | No |
| C40H80N1O8P1 | [M + H]⁺ | 734.56943 | PC(16:0_16:0) | Yes | No |
| C57H100O6 | [M + Na]⁺ | 903.74121 | TAG(54:6) | Yes | Yes |
| C24H59N1O7P1 | [M + H]⁺ | 496.33977 | LysoPC(16:0) | Yes | No |
| C20H42N1O6P1 | [M + H]⁺ | 424.28225 | LysoPC-pmg(12:0)⁴ | Yes | No |

¹Molecular formula
²estimated mass error >±0.1 ppm. These lipid features (as defined in the text) were verified by further MS1 analysis as described in the Methods, except that the resolving power was set to 500,000 at m/z = 200.
³In this study lipid features (monoisotopic accurate m/z values) were used for classification. The molecular formulae and lipid name assignments are interpretations listed for the reader. The assignments were based on accurate mass and MS2 fragmentation patterns. CE, cholesterol esters; TAG, triacylglyceride; LysoPC-pmg, lysophosphatidylcholine-plasmalogen; PC phosphatidyl choline; SM sphingomyelin. Nomenclature according to [82, 83].
⁴This molecular formula and lipid assignment was the closest from our comprehensive lipids database. Among non-lipids there is the possibility of the non-phosphate ³¹P-containing compound C19H37N8O1P1, which is inconsistent with the phosphocholine fragment in the MS2 data (FIG. 14).

### Discussion and Conclusions

Two orthogonal multivariate statistical tools (RF and LASSO) have been applied to classify different stages of NSCLC versus normal individuals based on UHR-FTMS analysis of lipid profiles of plasma exosomes from peripheral blood, a form of “liquid biopsy”. The data sets were large and highly sparse with many zero values and a high dynamic range, making accurate classification difficult by the classical methods (cf. FIG. 11). Using our in-house program PREMISE, we were able to assign 430 lipids by class, and their importance to the classification was determined using the Gini importance. For the RF method, these enabled the choice of 16 lipids for the final classification, which gave good AUROCs with reasonable sensitivity and specificity indices for discriminating normal subjects from early and late stage NSCLC patients (Table 6). In comparison, the LASSO method selected 7 lipid features for classification, which gave somewhat lower AUROC values but higher specificity indices for the same types of classification. It is also interesting to note that three of the validated lipid species overlapped between the two methods (Table 7), which added confidence to their utility in classifying NSCLC.

The final data sets were scrutinized at multiple levels of quality control, i.e. at the sample collection/processing level as well as subsequent MS data and multivariate statistical analyses. We emphasize the importance of removing contaminants/spectral artifacts and exploring normalization of the MS raw data for subsequent statistical analysis. Initial analysis by RF and LASSO without adequate correction for solvent impurities and Orbitrap spectral artifacts gave unreasonably high AUROC values of close to 1.0 for both methods. Some of the important classifiers turned out to be solvent impurities and spectral artifacts. After extensive investigations including manual curation and multiple iterations of artifact corrections and different normalization methods, we confirmed the lipid identity of all 16 features of the RF method and 3 of the LASSO method, which should greatly improve the accuracy of the classification. Since the RF and LASSO methods are independent approaches, the congruence of the two methods afforded greater confidence in the result. We consider this combined statistical approach with extensive quality control to be a step forward in biomarker analysis for complex and sparse datasets.

It should be noted that the majority of our lung cancer cohorts were smokers and many with some forms of inflammatory co-morbidities such as chronic obstructive pulmonary disease (COPD). COPD is considered to be a high risk factor for lung cancer development. Also noted is the moderate number of subjects used for this report. The next step is to increase the study size with a blinded validation set to assess the overall accuracy for NSCLC classification and to determine exosomal lipid classifiers for discrimination of COPD or other inflammatory lung diseases from early stage lung cancer. We have begun collecting samples from subjects with COPD without lung cancer to use in the methods disclosed herein.

In conclusion, both RF and LASSO-based multivariate statistical analyses of plasma exosomal lipid profiles were highly informative in discriminating normal from early and late stage lung cancer subjects with a moderate study size. The selected and validated lipid classifiers (e.g. SM and LPC) may not only be useful as lung cancer biomarkers but could also have important functions in exosome biogenesis and immune cell interactions.

Throughout this document, various references are mentioned. All such references are incorporated herein by reference, including the references set forth in the following list:

### REFERENCES

1. Higashi, R. M. (2011) In Fan, T. W Higashi, R. M., Lane, A. N. (ed.), Handbook of Metabolomics Methods. Humana Press, New York.
2. Lane, A. N., Fan, T. W. M., Xie, X., Moseley, H. N. and Higashi, R. M. (2009) Stable isotope analysis of lipid, biosynthesis by high resolution mass spectrometry and NMR Anal. Chim. Acta, 651, 201-2081
3. Tibshirani, R. (1996) Regression shrinkage and selection via the lasso. J. Royal Statistical Society. Series B (Methodological), 267-288.
4. Shi, T., Seligson D., Belldegrun A S., Palotie A and Horvath, S. (2005) Tumor classification by tissue microarray profiling: random forest clustering applied to renal cell carcinoma. Modern Pathology 18, 47-557
5. Zhu, L., Wang, K., Cui, J., Liu, H., Bu, X., Ma, H., Wang, W., Gong, H., Lausted, C., Hood, L. et al. (2014) Label-Fre quantitative Detection of Tumor-Derived Exosomes through Surface Plasmon Resonance Imaging. Analytical Chemistry, 86, 8857-8864.
6. Im, H., Shao, H., Park, Y. I., Peterson, V. M., Castro, C. M., Weissleder, R. and Lee, H. (2014) Label-free detection and molecular profiling of exosomes with a nano-plasmonic sensor. Nature Biotechnology, 32, 490-U219.
7. Peterson, V. M., Castro, C. M., Chung, J., Miller, N. C., Ullal, A. V., Castano, M. D., Penson, R. T., Lee, H., Birrer, and Weissleder, R. (2013) Ascites analysis by a microfluidic chip allows tumor-cell profiling. Proceedings of the National Academy of Sciences of the United States of America, 110, E4978-E4986.
8. Rho, J., Chung, J., Im, H., Liong, M., Shao, H., Castro, C. M., Weissleder, R. and Lee, H. (2013) Magnetic posensor for Detection and Profiling of Erythrocyte-Derived Microvesicles. Acs Nano, 7, 11227-11233.
9. Berman, D. M.; Karhadkar, S. S.; Hallahan, A. R.; Pritchard, J. I.; Eberhart, C. G.; Watkins, D. N.; Chen, J. K.; Cooper, M. K.; Taipale, J.; Olson, J. M.; Beachy, P. A. Science 2002, 297, 1559-1561.
10. Rudin, C. M.; Hann, C. L.; Laterra, J.; Yauch, R. L.; Callahan, C. A.; Fu, L.; Holcomb, T.; Stinson, J.; Gould, S. E.; Coleman, B.; LoRusso, P. M.; Hoff, Von, D. D.; de Sauvage, F. J.; Low, J. A. N Engl J Med 2009, 361, 1173-1178.
11. Rosow, D. E.; Liss, A. S.; Strobel, O.; Fritz, S.; Bausch, D.; Valsangkar, N. P.; Alsina, J.; Kulemann, B.; Park, J. K.; Yamaguchi, J.; LaFemina, J.; Thayer, S. P. Surgery 2012, 152, S19-S32.
12. Marien E, Meister M, Muley T, Fieuws S, Bordel S, Derua R, Spraggins J, Van de Plas R, Dehairs J, Wouters J, Bagadi M, Dienemann H, Thomas M, Schnabel P A, Caprioli R M, Waelkens E, Swinnen J V. Non-small cell lung cancer is characterized by dramatic changes in phospholipid profiles. Int J Cancer. Oct. 1, 2015; 137(7):1539-48.
13. Patel N, Vogel R, Chandra-Kuntal K, Glasgow W, Kelavkar U. A novel three serum phospholipid panel differentiates normal individuals from those with prostate cancer. PLoS One. Mar. 6, 2014; 9(3):e88841.
14. Elham Hosseini-Beheshti, Steven Pham, Hans Adomat, Na Li, and Emma S. Tomlinson Guns. Exosomes as Biomarker Enriched Microvesicles: Characterization of Exosomal Proteins Derived from a Panel of Prostate Cell Lines with Distinct A R Phenotypes. Mol Cell Proteomics. October 2012; 11(10): 863-885.
15. Howarth D R, Lum S S, Esquivel P, Garberoglio C A, Senthil M, Solomon N L. Initial Results of Multigene Panel Testing for Hereditary Breast and Ovarian Cancer and Lynch Syndrome. Am Surg. October 2015; 81(10):941-4.
16. Holohan C, Van Schaeybroeck S, Longley D B and Johnston P G (2013) Cancer drug resistance: an evolving paradigm. Nature Reviews Cancer 13, 714-726.
17. International Patent Application Publication No. WO 2011/163332 for “Methods for detecting cancer.”
18. M. J. Hayat, N. Howlader, M. E. Reichman, B. K. Edwards, Cancer statistics, trends, and multiple primary cancer analyses from the surveillance, epidemiology, and end results (SEER) program, Oncologist, 12 (2007) 20-37.
19. R. Siegel, E. Ward, O. Brawley, A. Jemal, Cancer statistics, 2011, C A: a cancer journal for clinicians, 61 (2011) 212-236.
20. R. L. Siegel, K. D. Miller, A. Jemal, Cancer statistics, 2016, C A: a cancer journal for clinicians, 66 (2016) 7-30.
21. Hopenhayn C, Jenkins T M, P. J., The burden of lung cancer in Kentucky, J Ky Med Assoc., 101 (2003) 15-20.
22. A. K. Greenberg, M. S. Lee, Biomarkers for lung cancer: clinical uses, Curr Opin Pulm Med, 13 (2007) 249-255.
23. M. J. Hayat, N. Howlader, M. E. Reichman, B. K. Edwards, Cancer statistics, trends, and multiple primary cancer analyses from the Surveillance, Epidemiology, and End Results (SEER) Program, Oncologist, 12 (2007) 20-37.
24. M. Unger, A Pause, Progress, and Reassessment in Lung Cancer Screening, N Engl Med, 355 (2006) 1822-1824.
25. L. G. Collins, C. Haines, R. Perkel, R. E. Enck, Lung cancer: Diagnosis and management, American Family Physician, 75 (2007) 56-63.
26. D. R. Aberle, A. M. Adams, C. D. Berg, W. C. Black, J. D. Clapp, R. M. Fagerstrom, I. F. Gareen, C. Gatsonis, P. M. Marcus, J. D. Sicks, Reduced lung-cancer mortality with low-dose computed tomographic screening, The New England journal of medicine, 365 (2011) 395-409.
27. J. D. Campbell, A. Alexandrov, J. Kim, J. Wala, A. H. Berger, C. S. Pedamallu, S. A. Shukla, G. W. Guo, A. N. Brooks, B. A. Murray, M. Imielinski, X. Hu, S. Y. Ling, R. Akbani, M. Rosenberg, C. Cibulskis, A. Ramachandran, E. A. Collisson, D. J. Kwiatkowski, M. S. Lawrence, J. N. Weinstein, R. G. W. Verhaak, C. J. Wu, P. S. Hammerman, A. D. Chemiack, G. Getz, M. N. Artyomov, R. Schreiber, R. Govindan, M. Meyerson, N. Canc Genome Atlas Res, Distinct patterns of somatic genome alterations in lung adenocarcinomas and squamous cell carcinomas, Nature Genetics, 48 (2016) 607-+.
28. E. A. Collisson, et al., Canc Genome Atlas Res, Comprehensive molecular profiling of lung adenocarcinoma, Nature, 511 (2014) 543-550.
29. M. Imielinski, et al., Mapping the Hallmarks of Lung Adenocarcinoma with Massively Parallel Sequencing, Cell, 150 (2012) 1107-1120.
30. P. S. Hammerman, et al. Canc Genome Atlas Res, Comprehensive genomic characterization of squamous cell lung cancers, Nature, 489 (2012) 519-525.
31. F. Skoulidis, L. A. Byers, L. X. Diao, V. A. Papadimitrakopoulou, P. Tong, J. Izzo, C. Behrens, H. Kadara, E. R. Parra, J. R. Canales, J. J. Zhang, U. Giri, J. Gudikote, M. A. Cortez, C. Yang, Y. H. Fan, M. Peyton, L. Girard, K. R. Coombes, C. Toniatti, T. P. Heffernan, M. Choi, G. M. Frampton, V. Miller, J. N. Weinstein, R. S. Herbst, K. K. Wong, J. H. Zhang, P. Sharma, G. B. Mills, W. K. Hong, J. D. Minna, J. P. Allison, A. Futreal, J. Wang, Wistuba, II, J. V. Heymach, Co-occurring Genomic Alterations Define Major Subsets of KRAS-Mutant Lung Adenocarcinoma with Distinct Biology, Immune Profiles, and Therapeutic Vulnerabilities, Cancer Discovery, 5 (2015) 860-877.
32. D. Hanahan, R. A. Weinberg, Hallmarks of Cancer: The Next Generation, Cell, 144 (2011) 646-674.
33. A. N. Lane, T. W. M. Fan, M. Bousamra. R. M. Higashi, J. Yan, D. M. Miller, Stable Isotope-Resolved Metabolomics (SIRM) in Cancer Research with Clinical Applications of Non-Small Cell Lung Cancer, Omics, 15 (2011) 173-182.
34. K. Zaugg, Y. Yao, P. T. Reilly, K. Kannan, R. Kiarash, J. Mason, P. Huang, S. K. Sawyer, B. Fuerth, B. Faubert, T. Kalliomaki, A. Elia, X. Luo, V. Nadeem, D. Bungard, S. Yalavarthi, J. D. Growney, A. Wakeham, Y. Moolani, J. Silvester, A. Y. Ten, W. Bakker, K. Tsuchihara, S. L. Berger, R. P. Hill, R. G. Jones, M. Tsao, M. O. Robinson, C. B. Thompson, G. Pan, T. W. Mak, Camitine palmitoyltransferase 1C promotes cell survival and tumor growth under conditions of metabolic stress, Genes Dev, 25 (2011) 1041-1051.
35. S. Beloribi-Djefaflia, S. Vasseur, F. Guillaumond, Lipid metabolic reprogramming in cancer cells, Oncogenesis, 5 (2016) e189.
36. V. Muralidharan-Chari, J. W. Clancy, A. Sedgwick, C. D'Souza-Schorey, Microvesicles: mediators of extracellular communication during cancer progression, Journal of Cell Science, 123 (2010) 1603-1611.
37. D. Zech, S. Rana, M. W. Buehler, M. Zoller, Tumor-exosomes and leukocyte activation: an ambivalent crosstalk, Cell Commun Signal, 10 (2012) 37.
38. J. Rak, Microparticies in Cancer, Seminars in Thrombosis and Hemostasis, 36 (2010) 888-906.
39. C. Liu, S. Yu, K. Zinn, J. Wang, L. Zhang, Y. Jia, J. C. Kappes, S. Barnes, R. P. Kimberly, W. E. Grizzle, H. G. Mang, Murine mammary carcinoma exosomes promote tumor growth by suppression of N K cell function, Journal of immunology, 176 (2006) 1375-1385.
40. J. Couzin, Cell biology: The ins and outs of exosomes, Science, 308 (2005) 1862-1863.
41. M. Wysoczynski, M. Z. Ratajczak, Lung cancer secreted microvesicles: underappreciated modulators of microenvironment in expanding tumors, International journal of cancer. Journal international du cancer, 125 (2009) 1595-1603.
42. A. Janowska-Wieczorek, M. Wysoczynski, J. Kijowski, L. Marquez-Curtis, B. Machalinski, J. Ratajczak, M. Z. Ratajczak, Microvesicles derived from activated platelets induce metastasis and angiogenesis in lung cancer, Int J Cancer, 113 (2005) 752-760.
43. J. Skog, T. Wurdinger, S. van Rijn, D. H. Meijer, L. Gainche, M. Sena-Esteves, W. T. Curry, Jr., B. S. Carter, A. M. Krichevsky, X. O. Breakefield, Glioblastoma microvesicles transport RNA and proteins that promote tumour growth and provide diagnostic biomarkers, Nat Cell Biol, 10 (2008) 1470-1476.
44. W. T. Arscott, K. A. Camphausen, EGFR isoforms in exosomes as a novel method for biomarker discovery in pancreatic cancer, Biomark Med, 5 (2011) 821.
45. S. Gesierich, I. Berezovskiy, E. Ryschich, M. Zoller, Systemic induction of the angiogenesis switch by the tetraspanin D6.1A/CO-029, Cancer Res, 66 (2006) 7083-7094.
46. B. Costa-Silva, N. M. Aiello, A. J. Ocean, S. Singh, H. Zhang, B. K. Thakur, A. Becker, A. Hoshino, M. T. Mark, H. Molina, I Xiang, T. Zhang, T. M. Theilen, G. Garcia-Santos, C. Williams, Y. Ararso, Y. Huang, G. Rodrigues, T. L. Shen, K. J. Labori, I. M. B. Lothe, E. H. Kure, I Hernandez, A. Doussot, S. H. Ebbesen, P. M. Grandgenett, M. A. Hollingsworth, M. Jain, K. Mallya, S. K. Batra, W. R. Jarnagin, R. E. Schwartz, I. Matei, H. Peinado, B. Z. Stanger, J. Bromberg, D. Lyden, Pancreatic cancer exosomes initiate pre-metastatic niche formation in the liver, Nat Cell Biol, 17 (2015) 816-826.
47. A. Hoshino, B. Costa-Silva, T. L. Shen, G. Rodrigues, A. Hashimoto, M. Tesic Mark, H. Molina, S. Kohsaka, A. Di Giannatale, S. Ceder, S. Singh, C. Williams, N. Soplop, K. Uryu, L. Pharmer, T. King, L. Bojmar, A. E. Davies, Y. Ararso, T. Mang, H. Zhang, J. Hernandez, J. M. Weiss, V. D. Dumont-Cole, K. Kramer, L. H. Wexler, A. Narendran, G. K. Schwartz, J. H. Healey, P. Sandstrom, K. J. Labori, E. H. Kure, Grandgenett, M. A. Hollingsworth, M. de Sousa, S. Kaur, M. Jain, K. Mallya, S. K. Batra, W. R. Jamagin, M. S. Brady, O. Fodstad, V. Muller, K. Pantel, A. J. Minn, M. J. Bissell, B. A. Garcia, Y. Kang, V. K. Rajasekhar, C. M. Ghajar, I. Matei, H. Peinado, J. Bromberg, D. Lyden, Tumour exosome integrins determine organotropic metastasis, Nature, 527 (2015) 329-335.
48. M. Frydrychowicz, A. Kolecka-Bednarczyk, M. Madejczyk, S. Yasar, G. Dworacki, Exosomes—structure, biogenesis and biological role in non-small-cell lung cancer, Scandinavian journal of immunology, 81 (2015) 2-10.
49. R. Safaei, B. J. Larson, T. C. Cheng, M. A. Gibson, S. Otani, W. Naerdemann, S. B. Howell, Abnormal lysosomal trafficking and enhanced exosomal export of cisplatin in drug-resistant human ovarian carcinoma cells, Mol Cancer Ther, 4 (2005) 1595-1604.
50. D. D. Yu, Y. Wu, H. Y. Shen, M. M. Lv, W. X. Chen, X. H. Zhang, S. L. Zhong, J. H. Tang, J. H. Zhao, Exosomes in development, metastasis and drug resistance of breast cancer, Cancer science, 106 (2015) 959-964.
51. M. A. Rahman, J. F. Barger, F. Lovat, M. Gao, G. A. Otterson, P. Nana-Sinkam, Lung cancer exosomes as drivers of epithelial mesenchymal transition, Oncotarget, (2016).
52. X. Xiao, S. Yu, S. Li, J. Wu, R. Ma, H. Cao, Y. Zhu, J. Feng, Exosomes: decreased sensitivity of lung cancer A549 cells to cisplatin, PLoS One, 9 (2014) e89534.
53. S. Beloribi, E. Ristorcelli, G. Breuzard, F. Silvy, J. Bertrand-Michel, E. Beraud, A. Verine, D. Lombardo, Exosomal lipids impact notch signaling and induce death of human pancreatic tumoral SOJ-6 cells, PLoS One, 7 (2012) e47480.
54. S. Beloribi-Djefaflia, C. Siret, D. Lombardo, Exosomal lipids induce human pancreatic tumoral MiaPaCa-2 cells resistance through the CXCR4-SDF-1alpha signaling axis, Oncoscience, 2 (2015) 15-30.
55. I. Parolini, C. Federici, C. Raggi, L. Lugini, S. Palleschi, A. De Milito, C. Coscia, E. Iessi, M. Logozzi, A. Molinari, M. Colone, M. Tatti, M. Sargiacomo, S. Fais, Microenvironmental pH is a key factor for exosome traffic in tumor cells, The Journal of biological chemistry, 284 (2009) 34211-34222.
56. M. P. Plebanek, R. K. Mutharasan, O. Volpert, A. Matov, J. C. Gatlin, C. S. Thaxton, Nanoparticle Targeting and Cholesterol Flux Through Scavenger Receptor Type B-1 Inhibits Cellular Exosome Uptake, Scientific reports, 5 (2015) 15724.
57. A. Carracedo, M. Gironella, M. Lorente, S. Garcia, M. Guzman, G. Velasco, J. L. Iovanna, Cannabinoids induce apoptosis of pancreatic tumor cells via endoplasmic reticulum stress-related genes, Cancer Res, 66 (2006) 6748-6755.
58. B. Madhavan, S. Yue, U. Galli, S. Rana, W. Gross, M. Muller, N. A. Giese, H. Kalthoff, T. Becker, M. W. Buchler, M. Zoller, Combined evaluation of a panel of protein and miRNA serum-exosome biomarkers for pancreatic cancer diagnosis increases sensitivity and specificity, Int J Cancer, 136 (2015) 2616-2627.
59. S. Komatsu, D. Ichikawa, H. Takeshita, R. Morimura, S. Hirajima, M. Tsujiura, T. Kawaguchi, M. Miyamae, H. Nagata, H. Konishi, A. Shiozaki, E. Otsuji, Circulating miR-18a: a sensitive cancer screening biomarker in human cancer, In vivo (Athens, Greece), 28 (2014) 293-297.
60. M. Zoller, Pancreatic cancer diagnosis by free and exosomal miRNA, World J Gastrointest Pathophysiol, 4 (2013) 74-90.
61. R. Que, G. Ding, J. Chen, L. Cao, Analysis of serum exosomal microRNAs and clinicopathologic features of patients with pancreatic adenocarcinoma, World J Surg Oncol, 11 (2013) 219.
62. K. R. Jakobsen, B. S. Paulsen, R. Bæk, K. Vanning, B. S. Sorensen, M. M. Jorgensen, Exosomal proteins as potential diagnostic markers in advanced non-small cell lung carcinoma, Journal of Extracellular Vesicles, 4 (2015) 10.3402/jev.v3404.26659.
63. G. Rabinowits, C. Gercel-Taylor, J. M. Day, D. D. Taylor, G. H. Kloecker, Exosomal microRNA: a diagnostic marker for lung cancer, Clin Lung Cancer, 10 (2009) 42-46.
64. T. W. M. Fan, Sample Preparation for Metabolomics Investigation, in: T. W. M. Fan, A. N. Lane, R. M. Higashi (Eds.) The Handbook of Metabolomics: Pathway and Flux Analysis, Methods in Pharmacology and Toxicology. DOI 10.1007/978-1-61779-618-0_11, Springer Science, New York, 2012, pp. 7-27.
65. A. N. Lane, T. W. Fan, R. M. Higashi, Isotopomer-based metabolomic analysis by NMR and mass spectrometry, Methods Cell Biol, 84 (2008) 541-588.
66. W. J. Carreer, R. M. Flight, H. N. Moseley, A Computational Framework for High-Throughput Isotopic Natural Abundance Correction of Omics-Level Ultra-High Resolution FT-MS Datasets, Metabolites, 3 (2013).
67. H. N. Moseley, Correcting for the effects of natural abundance in stable isotope resolved metabolomics experiments involving ultra-high resolution mass spectrometry, BMC Bioinformatics, 11 (2010) 139.
68. L. Breiman, Random forests, Machine Learning, 45 (2001) 5-32.
69. Y. Qi, Random Forest for Bioinformatics, in: Mang C., M. Y. (Eds.) Ensemble Machine Learning, Springer, Boston, 2012, pp. 307-323.
70. R. Tibshirani, Regression shrinkage and selection via the lasso., J. Royal Statistical Society. Series B (Methodological) (1996) 267-288.
71. B. Worley, R. Powers, Multivariate Analysis in Metabolomics, Current Metabolomics, 1 (2013) 92-107.
72. C. Subra, K. Laulagnier, B. Perret, M. Record, Exosome lipidomics unravels lipid sorting at the level of multivesicular bodies, Biochimie, 89 (2007) 205-212.
73. R. Siegel, E. Ward, O. Brawley, A. Jemal, Cancer statistics, 2011, C A: a cancer journal for clinicians, 61 (2011) 212-236.
74. R. L. Siegel, K. D. Miller, A. Jemal, Cancer statistics, 2016, C A: a cancer journal for clinicians, 66 (2016) 7-30.
75. M. Unger, A Pause, Progress, and Reassessment in Lung Cancer Screening, N Engl J Med, 355 (2006) 1822-1824.
76. L. G. Collins, C. Haines, R. Perkel, R. E. Enck, Lung cancer: Diagnosis and management, American Family Physician, 75 (2007) 56-63.
77. D. R. Aberle, A. M. Adams, C. D. Berg, W. C. Black, J. D. Clapp, R. M. Fagerstrom, Gareen, C. Gatsonis, P. M. Marcus, J. D. Sicks, Reduced lung-cancer mortality with low-dose computed tomographic screening, The New England journal of medicine, 365 (2011) 395-409.
78. T. Baranyai, K. Herczeg, Z. Onodi, I. Voszka, K. Modos, N. Marton, G Nagy, I. Maeger, M. J. Wood, S. El Andaloussi, Z. Palinkas, V. Kumar, P. Nagy, A. Kittel, E. I. Buzas, P. Ferdinandy, Z. Giricz, Isolation of Exosomes from Blood Plasma: Qualitative and Quantitative Comparison of Ultracentrifugation and Size Exclusion Chromatography Methods, Plos One, 10 (2015).
79. K. Koga, K. Matsumoto, T. Akiyoshi, M. Kubo, N. Yamanaka, A. Tasaki, H. Nakashima, M. Nakamura, S. Kurok, M. Tanaka, M. Katano, Purification, characterization and biological significance of tumor-derived exosomes, Anticancer Research, 25 (2005) 3703-3707.
80. B. H. Menze, B. M. Kelm, R. Masuch, U. Himmelreich, P. Bachert, W. Petrich, Hamprecht, A comparison of random forest and its Gini importance with standard chemometric methods for the feature selection and classification of spectral data, BMC Bioinformatics, 10 (2009) 213.
81. R. Tibshirani, Regression shrinkage and selection via the lasso., J. Royal Statistical Society., Series B (Methodological) (1996) 267-288.
82. E. Fahy, S. Subramaniam, R. C. Murphy, M. Nishijima, C. R. H. Raetz, T. Shimizu, F. Spener, G. van Meer, M. J. O. Wakelam, E. A. Dennis, Update of the LIPID MAPS comprehensive classification system for lipids, Journal of Lipid Research, 50 (2009) S9-S14.
83. G. Liebisch, J. A. Vizcaíno, H. Köfeler, M. Trötzmiiller, W. J. Griffiths, G. Schnitz, F. Spener, M. J. O. Wakelam, Shorthand notation for lipid structures derived from mass spectrometry, J. Lipid Res., 54 (2013) 1523-1530.

We claim:

1. A method for determining amounts of lipids in a sample from a subject suspected of having lung cancer, comprising:
providing a biological sample selected from the group consisting of a blood sample, a serum sample, and a plasma sample from the subject suspected of having lung cancer;
isolating exosomes from the biological sample;
extracting lipids from the exosomes to obtain exosomal lipids;
determining using mass spectrometry the amount of lipids present in the exosomal lipids in a lipid set comprising five lipids;
wherein one of the five lipids is selected from the group consisting of phosphatidyl choline (PC) (18:2/18:1), PC (18:2/18:0), PC (22:6/16:0), PC (18:2/16:0), PC (16:0/16:0), PC (18:1/18:0), PC (20:3/18:0), PC (20:4/16:0), and PC (22:5/16:0);
wherein the remaining four lipids of the five lipids are selected from the group consisting of PC (36:2), PC (36:1), PC (32:0), PC (18:2/18:1), PC (18:2/18:0), PC (22:6/16:0), PC (18:2/16:0), PC (16:0/16:0), PC (18:1/18:0), PC (20:3/18:0), PC (20:4/16:0), PC (22:5/16:0), sphingomyelin (SM) (18:1/16:0), SM (18:1/24:1), SM (18:1/16:0); triacylglyceride (TAG) (18:2/16:0/20.4), TAG (18:1/18:2/16:2), cholesterol ester (CE) (20:4), lysophosphatidylcholine (LysoPC) (16:0), and lysophosphatidylcholine-plasmalogen (LysoPC-pmg) (12:0).

2. A method for determining amounts of lipids in a sample from a subject suspected of having cancer, comprising:
providing a biological sample from the subject suspected of having cancer;
isolating exosomes from the biological sample;
extracting lipids from the exosomes to obtain exosomal lipids;
determining using mass spectrometry the amount of lipids present in the exosomal lipids in a lipid set comprising a plurality of lipids;
wherein five lipids of the lipid set are selected from the group consisting of phosphatidyl choline (PC) (18:2/18:1), PC (18:2/18:0), PC (22:6/16:0), PC (18:2/16:0), PC (16:0/16:0), PC (18:1/18:0), PC (20:3/18:0), PC (20:4/16:0), and PC (22:5/16:0).
